(19) United States
(12) Patent Application Publication
(10) Pub. No.: US 2017/0088812 A1
(43) Pub. Date: Mar. 30, 2017

(54) DECREASED LIGHT-HARVESTING ANTENNA SIZE IN CYANOBACTERIA

(71) Applicant: The Regents of The University of California, Oakland, CA (US)

(72) Inventors: Anastasios Melis, El Cerrito, CA (US); Henning Kirst, Burgwedel (DE)

(21) Appl. No.: 15/312,615

(22) PCT Filed: May 21, 2015
(86) PCT No.: PCT/US15/31924

§ 371 Date: Nov. 18, 2016

Related U.S. Application Data
(60) Provisional application No. 62/001,326, filed on May 21, 2014.

(51) Int. Cl. C12N 1/20; C12N 13/00

(52) U.S. Cl. CPC C12N 1/20; C12N 13/00; C12N 2510/02

(57) ABSTRACT

The invention provides methods and compositions for increasing photosynthetic efficiency and biomass production in cyanobacterial cultures by minimizing the phycobilisome light-harvesting antenna size through disruption of the phycocyanin-encoding CPC-operon.

### CROSS-REFERENCE TO RELATED APPLICATIONS

This application claims priority benefit of U.S. provisional application No. 62/001,326, filed May 21, 2014, which application is herein incorporated by reference.

### STATEMENT AS TO RIGHTS TO INVENTIONS MADE UNDER FEDERALLY SPONSORED RESEARCH AND DEVELOPMENT

This invention was made with Government support under Grant No. DE-FG36-05GO15041 awarded by the Department of Energy. The Government has certain rights in this invention.

### REFERENCE TO SUBMISSION OF A SEQUENCE LISTING

This application includes a Sequence Listing as a text file named “SEQ_86540-943406” created May 21, 2015 and containing 15.174 bytes. The material contained in this text file is incorporated by reference in its entirety for all purposes.

### BACKGROUND OF THE INVENTION

Cyanobacteria have evolved an auxiliary light-harvesting system, the phycobilisome (PBS) that allows absorption of sunlight, primarily in the 575-675 nm region, and unidirectional excitation energy transfer toward the chlorophyll-pigment bed of PSII reaction centers. Each phycobilisome has two main structural parts, the core-cylinders and the peripheral rods. Core cylinders are made of allophycocyanin (aβ)3 discs stacked next to each other. The core cylinders axis is parallel to the thylakoid membrane surface with at least two of the cylinders resting with their long axes on the stromal side of the thylakoid membrane. These provide a structural and excitation energy transfer link to the chlorophyll-pigment bed of PSII reaction centers (Elmorjani et al. 1986; Glazer and Melis 1987; Ducret et al. 1996; Glazer 1989; Arteni et al. 2009). In Synechocystis sp. PCC 6803 (Synechocystis), there are three allophycocyanin core cylinders, two of which rest directly onto the thylakoid membrane. A third cylinder is resting on the stromal side of the furrow formed by the other two core cylinders (Arteni et al. 2009). Core cylinders contain the pigment-proteins allophycocyanin-α and allophycocyanin-β encoded by the APCA and APCB genes and a small linker polypeptide LC, encoded by the APCC gene (Grossman et al. 1993; Glazer 1998; MacColl 1998). They are linked to the thylakoid membrane and the PSII dimer chlorophyll-pigments by a PBS terminal excitation-acceptor allophycocyanin pigment containing the linker polypeptide LCM, encoded by the APCE gene (Houmard et al. 1990; Ajlani and Vernotte 1998). The latter functions together with the products of the APCD and APCF genes to facilitate efficient excitation energy transfer from the phycobilisome toward the PSII reaction center (Ashby and Mullineaux 1999; Barber et al. 2003; Mullineaux 2008). Peripheral to the allophycocyanin core cylinders are phycocyanin-containing rods, also in cylinder form, physically extending outward from the allophycocyanin core cylinders (Glazer and Melis 1987; Glazer 1998; Arteni et al. 2009). Similar to the allophycocyanin, the phycocyanin rods are composed of stacked discs, each one made by six hetero-dimers of the pigment containing CPC-α and CPC-β proteins, encoded by the CPCA and CPCB genes respectively (Grossman et al. 1993; Glazer 1998; MacColl 1998). The CPC-α and CPC-dimers are connected by linker polypeptides, encoded by CPCC1, CPCC2, and CPCD genes (Grossman et al. 1993; Ughy and Ajlani 2004). Genes CPCA, CPCB, CPCC1, CPCC2 and CPCD are clustered in a single operon in Synechocystis, which is referred to as the C-phycocyanin (CPC)-operon.

The phycobilisome substantially increases the sunlight absorption cross-section of PSII (Glazer and Melis 1987; Glazer 1989), thereby countering a potential imbalance in excitation energy distribution due to the high PSI/PSII stoichiometric ratio in cyanobacteria (Melis and Brown 1980, Myers et al. 1980), and the fact that most of the chlorophyll is associated with PSI in these microorganisms (Manodori et al. 1984; Glazer and Melis 1987). Up to 450 phycocyanin (PC) and allophycocyanin (AP) pigments can be associated with the PBS in Synechocystis. This large light-harvesting antenna size confers a survival advantage in the wild, where cells grow under light-limiting conditions. Under direct sunlight, however, the rate of photon absorption far exceeds the rate with which photosynthesis can utilize them, and excess light-energy is dissipated by non-photochemical quenching (Miller et al. 2001, Kirilovsky 2007; Bailey and Grossman 2008; Kirilovsky and Kerfeld 2012). A soluble carotenoid binding protein (orange carotenoid protein, OCP) plays essential role in this process in Synechocystis. The quenching of maximal fluorescence increases from 25-30% in WT cells to 60%-70% in cells overexpressing the OCP (Kirilovsky and Kerfeld 2012). Wasteful dissipation of excess absorbed irradiance would result in a suboptimal sunlight energy conversion. Moreover, dissipation of excess absorbed energy would enhance the probability of photodamage and photoinhibition of photosynthesis (Melis 1999). As a result, the utmost measured sunlight-to-biomass energy conversion efficiencies of cyanobacterial photosynthesis were reported to be in the range of 1-2%, whereas the theoretical maximum is 8-10% (Melis 2009). This pitfall affects all photosynthetic organisms (Melis 2009). It was alleviated in green microalgae, upon minimizing the size of the chlorophyll light-harvesting antenna, effectively limiting the capacity of the photosystems to absorb sunlight. This prevented over-absorption of photons by individual cells, enabling deeper sunlight-penetration into the culture, and affording an opportunity for more cells to be productive, in effect raising photosynthetic productivity of the culture as a whole (Polle et al. 2003, Kirst et al. 2012a, Kirst et al. 2012b). This concept of increasing photosynthetic productivity of a mass-culture under direct sunlight upon minimizing the light-harvesting antenna size is known as the Truncated Light-harvesting Antenna (TLA) concept (Melis 2009; Melis 2012; Kirst and Melis 2014).

However, applicability of the TLA concept in photosynthetic cyanobacteria has been questioned. Indeed, it was reported that a targeted truncation of the phycobilisome light-harvesting antenna of the model cyanobacteria Synechocystis lowered, rather than increased, productivity (Page et al. 2012). This is the opposite to the substantially increased the productivity of TLA plants and algae. (See also Liberton et al 2013). Applicants have now discovered that TLA can be applied to cyanobacteria to increase photosynthetic productivity.

### BRIEF SUMMARY OF THE INVENTION

The current invention is based, in part, on the discovery that the TLA concept for improving photosynthetic efficiency and productivity of mass cultures can be achieved in cyanobacteria by minimizing the phycobilisome light-harvesting antenna size and culture the cyanobacteria under suitable conditions. Illustrative aspects of the invention are summarized in the current section. Thus, in one aspect, the invention relates to a method of culturing cyanobacteria to have increased photosynthetic productivity, the method comprising culture cyanobacteria having a disrupted CPC-operon and decreased phycobilisome antenna size under conditions in which photosynthetic efficiency is increased.

In some embodiments, the invention provides a method of enhancing photosynthetic productivity from a cyanobacteria, the method comprising: providing a cyanobacterial cell population in which the cyanobacteria are genetically modified to have a disruption in a CPC-operon; growing the cyanobacterial cell population in a reactor to obtain a cyanobacterial culture; maintaining the culture under conditions in which the light intensity is at least 500 micromol photons per square meter per second; and the culture absorbs at least 70% of the incoming light. In some embodiments, the light intensity is at least 800 micromol photons per square meter per second. In further embodiments, the culture absorbs at least 80%, or at least 90% of the incoming light. In some embodiments, the disruption in the CPC-operon is inhibition of a CPCA and/or CPCB gene. In addition embodiments, the disruption in the CPC-operon is inhibition of at least one CPCC1, CPCC2, and CPCD. In some embodiments, the disruption in the CPC-operon is a deletion of a CPCA, CPCB, CPCC1, CPCC2, and CPCD genes. In some embodiments the cyanobacteria are a species of a genus selected from the group consisting of Synechocystis, Synechococcus, Cyanothece, and Thermosynechococcus. In some embodiments, the cyanobacteria are a species of a genus of filamentous cyanobacteria, e.g., wherein the genus is selected from the group consisting of Arthrospira, Nostoc, and Anabaena.

### BRIEF DESCRIPTION OF THE DRAWINGS

FIG. 1: Genetic map of the Synechocystis CPC-operon and replacement of the CPC-coding region with the NPTI gene conferring a kanamycin resistance. The dashed lines indicate nucleotide regions of the Synechocystis genomic DNA used for homologous recombination; CPC-operon genes are shown. The NPTI gene is the kanamycin resistance gene shown in the figure.

FIG. 2: PCR-analysis of Synechocystis wild type and three independent Δcpc-transformants to test for insertion locus and DNA copy homoplasmy. Primer-sets a and b tested for the correct replacement of the CPC-operon with the NPTI gene. Expected product size for the wild type, using primer set a, was 4,683 nt. Expected product size for the Δcpc-transformants, using primer set a, was 2,126 nt. Expected product size for the wild type, using primer set b, was 4,973 nt. Expected product size for the Δcpc-transformants, using primer set b, was 2,416 nt. Primer-sets c through h were designed to test for homoplasmy by specifically amplifying the wild type genomic DNA. No PCR amplification product was obtained using genomic DNA from the Δcpc-transformants.

FIG. 3: Absorbance spectra of live Synechocystis wild type (WT) and Δcpc-transformant cultures. The Δcpc-transformants lacked the phycocyanin absorbance band peaking at 625 nm.

FIG. 4: Absorbance spectra of the membrane fraction of Synechocystis wild type (WT) and Δcpc-transformant strains.

FIG. 5: Absorbance spectra of the soluble fraction of Synechocystis wild type (WT) and Δcpc-transformant strains. The absorbance peak for phycocyanin (625 nm) is evident in the WT, whereas allophycocyanin absorbance peaks are most pronounced at 650 and 675 nm in the Δcpc-transformant strains.

FIG. 6: SDS-PAGE analysis of total protein extracts from Synechocystis wild type and Δcpc transformants. Phycocyanin α-subunit (CPC-α) and phycocyanin β-subunit (CPC-β) are migrating at around 17 and 13 kD, respectively, and are clearly absent from the Δcpc transformants. The NPTI protein with a molecular weight of 27 kD is highly abundant in the Δcpc transformants.

FIG. 7: Western blot analyses of protein extracts from Synechocystis wild type and Δcpc transformants. Specific polyclonal antibodies were raised against the D1 protein, the PSII reaction center D1/D2 heterodimer (PSII RC), the RbcL, ATP synthase β-subunit (ATP-β), and the orange carotenoid protein (OCP), and were used in this study.

FIG. 8: Light-saturation curves of photosynthesis obtained with the Synechocystis wild type and Δcpc-transformants. A higher light-intensity was needed to saturate photosynthesis in the Δcpc-transformants compared to the wild type. The half-saturation intensity of photosynthesis for the two strains was measured to be 220 μmol photons m⁻² s⁻¹ for the wild type and 500 μmol photons m⁻² s⁻¹ for the Δcpc transformants.

FIG. 9: Early-stage growth of Synechocystis wild type and Δcpc-transformants under low light conditions. A: Cell growth at 50 μmol photons m⁻² s⁻¹. B: Cell growth at 170 μmol photons m⁻² s⁻¹. The Δcpc-transformants showed a retarded growth under 50 μmol photons m⁻² s⁻¹, but they grew with a rate closer to that of the wild type under 170 μmol photons m⁻² s⁻¹.

FIG. 10: Cell duplication time as a function of growth intensity for Synechocystis wild type and Δcpc-transformants. The Δcpc-transformants showed a slower than wild type growth under low-light intensities but this difference was diminished as the growth irradiance increased. It was estimated that wild type and Δcpc transformants would reach the same cell duplication time of about 13 h at 800-1,000 μmol photons m⁻² s⁻¹ (dashed line).

FIG. 11: Biomass accumulation in batch cultures Synechocystis wild type and Δcpc transformants, grown under simulated bright sunlight conditions (2,000 mol photons m⁻² s⁻¹). Cultures were diluted with fresh growth media once they approached the end of the linear growth phase, i.e., at about 0.9-1.0 mg dcw mL⁻¹. The slope of the linear regressions defined the rate of biomass accumulation. This rate was always greater for the Δcpc transformants grown under these conditions compared to the wild type.

FIG. 12: Summary of biomass accumulation measurements from several batch cultures under simulated bright sunlight conditions (2,000 μmol photons m⁻² s⁻¹). The linear regression of the points represents the average rate of biomass accumulation of wild type (WT) and Δcpc transformants. A 57% greater productivity of the Δcpc transformants, relative to that of the wild type, was measured.

FIG. 13: Phycobilisome-chlorophyll antenna organization in the thylakoid of Synechocystis wild type and Δcpc transformants. Cyanobacteria may possess up to 850 phycocyanin (PC), allophycocyanin (AP), and chlorophyll (Ch1) molecules per unit photosynthetic apparatus.

### DETAILED DESCRIPTION OF THE INVENTION

### Definitions

The term “CPC-operon” as used herein refers to the genes in cyanobacteria that encode the polypeptide components of phycocyanin-containing rods. Phycocyanin-containing rods are composed of stacked discs, each one made up of six hetero-dimers of the phycocyanobilin pigment-containing CPC-α and CPC-β proteins. CPC-α and CPC-β dimers are connected by linker polypeptides. The genes in a CPC-operon are CPCA and CPCB, which encode CPC-α and CPC-β proteins, respectively; and genes that encode the linker polypeptides, e.g., CPCC1, CPCC2, and CPCD. A CPC-operon comprises a cluster of at least CPCA and CPCB, and in most species of cyanobacteria, the cluster includes the genes encoding the linker polypeptides. Thus, typically, the five genes, or four genes, e.g., if there is only one CPCC gene, are in a cluster at the same location. Sequences of genes in the CPC operon and proteins encoded by the genes are known in the art for various cyanobacteria species. Examples of Synechocystis sp. PCC 6803 CPC-operon protein sequences and the total genome sequence accession number are provided in Table 3. An illustrative nucleic acid sequence for the Synechocystis sp. PCC 6803 CPC-operon in provided in Example 1. Other illustrative cyanobacteria CPC operon gene and protein sequences are also provided in Table 3, which shows CPC-operon protein sequence accession numbers and the total genome accession number for Cyanothece sp. PCC 8801, Synechococcus elongatus sp. PCC 6301, Synechococcus sp. PCC 7002, and Thermosynechococcus elongates BP-1. The regions of the genome that encode the CPC-operon proteins can readily be identified by one of ordinary skill in the art by the genome notation and through the protein sequences.

A “CPC-operon nucleic acid” or “CPC-operon gene” as used herein include cyanobacterial homologs of the illustrative CPCA, CPCB, CPCC1, CPCC2, and CPCD sequences provided herein as examples. A CPC-operon protein refers to a protein encoded by a CPC-operon gene. Thus, a “CPCA gene” as used herein encodes a polypeptide that has at least 70% identity to a CPCA protein having an accession number shown in Table 3, e.g., at least 70% identity to a CPCA protein of SEQ ID NO:2. A “CPCB gene” as used herein encodes a polypeptide that has at least 70% identity to a CPCB protein having an accession number shown in Table 3, e.g., at least 70% identity to a CPCB protein of SEQ ID NO:3. A “CPCC1 gene” as used herein encodes a polypeptide that has at least 50% identity to a CPCC1 protein having an accession number shown in Table 3, e.g., at least 50% identity to a CPCC1 protein of SEQ ID NO:4. A “CPCC2 gene” as used herein encodes a polypeptide that has at least 50% identity to a CPCC2 protein having an accession number shown in Table 3, e.g., at least 50% identity to a CPCC2 protein of SEQ ID NO:5. A “CPCD gene” as used herein encodes a polypeptide that has at least 50% identity to a CPCD protein having an accession number shown in Table 3, e.g., at least 50% identity to a CPCD protein of SEQ ID NO:6.

The terms “nucleic acid” and “polynucleotide” are used synonymously and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones. Thus, nucleic acids or polynucleotides may also include modified nucleotides, that permit correct read through by a polymerase. “Polynucleotide sequence” or “nucleic acid sequence” may include both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses conservatively modified variants thereof (e.g., degenerate codon substitutions) and complementary sequences, as well as the sequence explicitly indicated. The nucleic acid may be DNA, both genomic and cDNA. RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc

The phrase “nucleic acid sequence encoding” refers to a nucleic acid that codes for an amino acid sequence of at least 5 contiguous amino acids within one reading frame. The amino acid need not necessarily be expressed when introduced into a cell or other expression system, but may merely be determinable based on the genetic code. Thus, a polynucleotide may encode a polypeptide sequence that comprises a stop codon or contains a changed frame so long as at least 5 contiguous amino acids within one reading frame. The nucleic acid sequences may include both the DNA strand sequence that is transcribed into RNA and the RNA sequence. The nucleic acid sequences include both the full-length nucleic acid sequences as well as fragments from the full-length sequences. It should be further understood that the sequence includes the degenerate codons of the native sequence or sequences, which may be introduced to provide codon preference in a specific host cell.

The term “promoter” or “regulatory element” refers to a region or sequence determinants located upstream or downstream from the start of transcription that are involved in recognition and binding of RNA polymerase and other proteins to initiate transcription. A “cyanobacteria promoter” is a promoter capable of initiating transcription in cyanobacteria cells. Such promoters need not be of cyanobacterial origin, for example, promoters derived from viruses, can be used in the present invention.

“Disruption of a CPC-operon” in the context of this invention refers to an inactivation, inhibition, or suppression of the expression of one or more genes within a CPC-operon that results in a cyanobacteria that is deficient in phycocyanin and has phycobilisome (PBS) light-harvesting antennae that are severely reduced in size compared to wild type. The terms “suppress”, “disrupt”, “inhibit”, or “inactivate” in the context of a CPC-operon gene as used herein encompasses modifications to one or more genes in the CPC-operon that result in the absence of the protein encoded by the gene in a cyanobacteria cell as well as aberrant protein expression, e.g., decreased protein expression or expression of a protein that has decreased function compared to the native protein, that results in reduced PBS attennae size compared to the antenna size of a counterpart wild-type cyanobacteria cell that has a native CPC-operon. A disruption in a CPC-operon gene in a cyanobacteria of the invention results in at least a 40% reduction in the phycobilisome size, typically at least a 50%, 60%, 70%, or an 80% reduction in the phycobilisome size, in comparison to the wild type cyanobacteria that has a native CPC-operon.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

As used herein, the term “genetically modified” refers to any change in the endogenous genome of a cyanobacteria cell compared to a wild-type cell. Thus, changes that are introduced through recombinant DNA technology and/or classical mutagenesis techniques are both encompassed by this term. The changes may involve protein coding sequences or non-protein coding sequences such as regulatory sequences as promoters or enhancers.

An “expression cassette” as used herein refers to a nucleic acid construct, which when introduced into a cyanobacterial host cell, alters transcription and/or translation of one or more endogenous gene and/or results in transcription and/or translation of a RNA or polypeptide, respectively. Antisense constructs or sense constructs that are not or cannot be translated are expressly included by this definition.

In the case of polynucleotides used to inhibit expression of an endogenous CPC-operon gene, the introduced sequence need not be perfectly identical to a sequence of the target endogenous gene. The introduced polynucleotide sequence will typically be at least substantially identical (as determined below) to the target endogenous CPC-operon gene sequence. Thus, an introduced “polynucleotide sequence from” a CPC-operon gene may not be identical to the target CPC-operon gene to be suppressed, but is functional in that it is capable of inhibiting expression of the target CPC-operon gene.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” in the context of polynucleotide or polypeptide sequences means that a polynucleotide or polypeptide comprises a sequence that has at least 50% sequence identity to a reference nucleic acid or polypeptide sequence. Alternatively, percent identity can be any integer from 40% to 100%. Exemplary embodiments include at least: 50%, 55%, 60%, 65%, 70%, 75%, 80%, 85%, 90%, 95%, or 99% compared to a reference sequence using the programs described herein; preferably BLAST using standard parameters, as described below. Accordingly, a CPC-operon gene sequence of the invention include nucleic acid sequences that encodes a CPC-operon protein that have at least 50%, 55%, 60%, 65%, 70%, 75%, 80%, 85%, 90%, 95%, or 99% identity, or more, to a CPC-operon protein identified by the Accession number provided in Table 3, e.g., a Synechocystis sp. CPC-operon protein of SEQ ID NO:2, 3, 4, 5, or 6.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

The term “isolated”, when applied to a nucleic acid or protein, denotes that the nucleic acid or protein is essentially free of other cellular components with which it is associated in the natural state. It is preferably in a homogeneous state and may be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest.

The term “reactor” as used herein refers to the vessel in which cyanobacteria are grown.

### Introduction

The present invention relates to cyanobacteria in which one or more genes in the CPC-operon are disrupted, i.e., cyanobacteria that are deficient in phycocyanin, resulting in decreased PBS antenna size. Such cyanobacteria can be cultured under conditions in which they exhibit improved photosynthetic productivity. Cyanobacteria disrupted in the CPC-operon are thus useful for many purposes, e.g., for production of lipid/hydrocarbons, for carbon sequestration, for biomass generation, and the like.

In the present invention, improved photosynthetic productivity in cyanobacteria lacking phycocyanin is manifested at certain light intensity (Photosynthetically Active Radiation (PAR), which as used here, refers to visible light between 400 and 700 nm), e.g., of at least 500 micromol photons per square meter per second or great, e.g., at least 800 micromol photons per square meter per second or greater (full sunlight=2,500 micromol photons per square meter per second). Light intensity is influenced by the dimensions of the reactor in which the cyanobacteria are grown. Further, improvided photosynthetic productivity is influenced by the density of the cyanobacteria biomass as further described below. Thus, cyanobacteria the density and light intensity are factors considered together in culturing cyanobacteria as described herein.

### Generating CPC-Operon-Deficient Cyanobacteria

The invention employs various routine recombinant nucleic acid techniques. Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel et al., eds., 1994-2011).

CPC-operon deficient cyanobacteria can be generated using methods well known in the art. These include mutagenesis techniques and targeted knockout or suppression of one or more genes in the CPC-operon; or inactivation of the entire CPC-operon, e.g., using homologous recombination to delete the operon and/or to replace it with another nucleic acid sequence, e.g., that encodes a selectable marker. In some embodiments, a CPCA or a CPCB gene may be disrupted. In some embodiments, a CPCC1, CPCC2, or CPCCD gene may be disrupted. In some embodiments, two, three, or four more genes in the CPC operon are disrupted. In some embodiments, all five genes of a CPC-operon are disrupted.

A CPC-operon that is targeted for disruption can be a Synechoccus sp. CPC-operon, or the CPC-operon in another cyanobacteria. CPC-operon sequences are well known in the art. Table 3 provides illustrative CPC protein accession numbers and the corresponding genomic DNA sequence for the organism. Accordingly, one of skill can readily employ mutagenesis strategies to disrupt one or more genes in the operon.

One or more CPC-operon genes can be disrupted, e.g., interrupted, truncated, or deleted using various techniques, including, but not limited to, insertional mutagenesis, meganuclease genome modification, and/or homologous recombination. For example, CPC-operon genes can be partially, substantially, or completely deleted, silenced, inactivated, or down-regulated by insertion of nucleic acid sequences that disrupt the function and/or expression of the gene. Alternatively, one or more CPC-operon genes may be deleted, either partially, substantially, or completely to eliminate function. In certain embodiments, a microorganism of interest may be engineered by site-directed homologous recombination to inactivate one or more CPC-operon genes. In still other embodiments, RNAi or antisense nucleic acids may be used to partially, substantially, or completely silence, inactivate, or down-regulate one or more CPC-operon genes. In some embodiments, a CPC operon promoter may be inactivated or otherwise modified to decrease or inhibit expression of CPC operon genes.

### Preparation of Recombinant Vectors

Recombinant DNA vectors suitable for transformation of cyanobacteria cells are employed in the methods of the invention. Preparation of suitable vectors and transformation methods are well known in the art. For example, a DNA sequence encoding a sequence to suppress expression of one or more CPC-operon genes (described in further detail below) or that encodes a sequences such as a selectable marker that is inserted into a CPC operon, will be combined with transcriptional and other regulatory sequences to direct expression in cyanobacteria.

Similarly, a vector to perform homologous recombination will include sequences required for homologous recombination, such as flanking sequences that share homology with the target CPC-operon for promoting homologous recombination (see, for example, Mes and Stal, Gene. 2005 Feb. 14; 346:163-71; and Mes and Doeleman, J Bacteriol. 2006 October; 188(20):7176-85. In one embodiment, the region of the cyanobacterial genome target for insertion can be a non-protein coding region, such as a CPC-operon promoter sequence. In other embodiments, the targeted region of the cyanobacterial genome can be one ore more CPC-protein-encoding gene to be disrupted.

Regulatory sequences incorporated into vectors that comprise sequences that are to be expressed in the modified cyanobacterial cell include promoters, which may be either constitutive or inducible. In some embodiments, a promoter can be used to direct expression of the inserted nucleic acids under the influence of changing environmental conditions. Examples of environmental conditions that may effect transcription by inducible promoters include anaerobic conditions, elevated temperature, or the presence of light. Promoters that are inducible upon exposure to chemicals reagents are also used to express the inserted nucleic acids. Other useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible promoters, such as those of the hsp70/dnaK genes (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element also can be, for example, a nitrate-inducible promoter, e.g., derived from the spinach nitrite reductase gene (Back et al., Plant Mol. Biol. 17:9 (1991)), or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)), or a light.

In some embodiments, the promoter may be from a gene associated with photosynthesis in the species to be transformed or another species. For example such a promoter from one species may be used to direct expression of a protein in transformed cyanobacteria cells. Suitable promoters may be isolated from or synthesized based on known sequences from other photosynthetic organisms. Preferred promoters are those for genes from other photosynthetic species, or other photosynthetic organism where the promoter is active in cyanobacteria.

In some embodiments, a promoter for a nucleic acid construct that is inserted into cyanobacteria to disrupt one or more CPC operon genes is a constitutive promoter. Examples of constitutive strong promoters for use in cyanobacteria include, for example, the psbDI gene or the basal promoter of the psbDII gene. Various other promoters that are active in cyanobacteria are also known. These include the light inducible promoters of the psbA and psbA3 genes in cyanobacteria. Other promoters that are operative in plants, e.g., promoters derived from plant viruses, such as the CaM V35S promoters, can also be employed in cyanobacteria. For a description of strong and regulated promoters, e.g., active in the cyanobacterium Anabaena sp. strain PCC 7120, see e.g., Elhai, FEMS Microbio Lett 114:179-184, (1993).

A vector will also typically comprise a marker gene that confers a selectable phenotype on cyanobacteria transformed with the vector. Such markers are known. For example, the marker may encode antibiotic resistance, such as resistance to chloramphenicol, kanamycin, G418, bleomycin, hygromycin, and the like.

Cell transformation methods and selectable markers for cyanobacteria are well known in the art (Wirth, Mol. Gen. Genet., 216(1): 175-7 (1989); Koksharova, Appl. Microbiol. Biotechnol., 58(2): 123-37 (2002); Thelwell et al., Proc. Natl. Acad. Sci. U.S.A., 95:10728-10733 (1998)).

Any suitable unicellular cyanobacteria may be employed to generate a cyanobacteria that is disrupted in a CPC operon gene. These include Synechococcus and Thermosynechococcus sp., e.g., Synechococcus sp. PCC 7002, Synechococcus sp. PCC 6301, and Thermosynechococcus elongates; as well as Synechocystis sp., such as Synechocystis sp. PCC 6803; and Cyanothece sp., such as PCC 8801. Filamentous cyanobacteria may also be engineered to disrupt the CPC-operon in accordance with this invention. Filamentous cyanobacteria that can be used include, e.g., Gloeocapsa sp., Nostoc sp., e.g., Nostoc sp. PCC 7120, and Nostoc sphaeroides; Anabaena sp., e.g., Anabaena variabilis and Arthrospira sp. (“Spirulina”), such as Arthrospira platensis and Arthrospira maxima. Cyanobacteria that are genetically modified in accordance with the invention to disrupt one or more CPC-operon genes may also contain other genetic modifications, e.g., modifications to the terpenoid pathway, to enhance production of a desirect compound.

In some embodiments, a construct to inactivate a CPC operon encodes an antibiotic resistance gene. Transformants may thus be cultured in selective media containing an antibiotic to which an untransformed host cell is sensitive. Cyanobacteria normally have up to 100 copies of identical circular DNA chromosomes in each cell. Successful transformation with a vector to disrupt a CPC-operon gene that an antibiotic resistance gene normally occurs in only one, or just a few, of the many cyanobacterial DNA copies. In some embodiments, cyanobacterial transformants are cultured under continuous selective pressure conditions (presence of antibiotic over many generations) to achieve DNA homoplasmy in the transformed host organism. One of skill in the art understands that the number of generations and length of time of culture varies depending on the particular culture conditions employed. Homoplasmy can be determined, e.g., by monitoring the DNA composition in the cells to determine the presence of wild-type copies of the cyanobacterial DNA.

“Achieving homoplasmy” refers to a quantitative replacement of most, e.g., 70% or greater, or typically all, wild-type copies of the cyanobacterial DNA in the cell with the transformant DNA copy that carries the disrupted CPC-operon. This is normally attained over time, under the continuous selective pressure (antibiotic) conditions applied, and entails the gradual during growth replacement of the wild-type copies of the DNA with the transgenic copies, until no wild-type copy of the cyanobacterial DNA is left in any of the transformant cells. Achieving homoplasmy is typically verified by quantitative amplification methods such as genomic-DNA PCR using primers and/or probes specific for the wild-type copy of the cyanobacterial DNA. Transgenic DNA is typically stable under homoplasmy conditions and present in all copies of the cyanobacterial DNA.

### Suppression of One or More CPC-Operon Genes

For inhibition of endogenous CPC-operon genes (e.g., by mutation, e.g., by homologous recombination to inactivate all or part of a gene, antisense, or sense suppression) one of skill will recognize that the inserted polynucleotide sequence need not be identical and may be “substantially identical” to a sequence of the gene from which it was derived. As explained below, these variants are specifically covered by this term.

CPC-operon nucleic acid sequences can be used to prepare expression cassettes useful for inhibiting or suppressing expression of one or more CPC-operon genes in cyanobacteria. For instance, siRNA, antisense, or ribozyme technology can be conveniently used.

For antisense expression, a nucleic acid segment from the desired gene is cloned and operably linked to a promoter such that the antisense strand of RNA will be transcribed. The expression cassette is then transformed into cyanobacteria and the antisense strand of RNA is produced. The antisense nucleic acid sequence transformed into cyanobacteria will be substantially identical to at least a portion of the endogenous gene or genes to be repressed. The sequence, however, does not have to be perfectly identical to inhibit expression. Thus, an antisense or sense nucleic acid molecule encoding only a portion of a CPC-operon gene can be useful for producing a cyanobacteria in which the CPC-operon is disrupted.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred. Sequences can also be longer, e.g., 1000 or 2000 nucleotides are greater in length.

Catalytic RNA molecules or ribozymes can also be used to inhibit expression of a CPC-operon gene. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs.

Another method of suppression is sense suppression (also known as co-suppression). Introduction of expression cassettes in which a nucleic acid is configured in the sense orientation with respect to the promoter has been shown to be an effective means by which to block the transcription of target genes.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 90% or 95% to absolute identity would be most preferred. As with antisense regulation, the effect should apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter-than full-length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

Endogenous gene expression may also be suppressed by means of RNA interference (RNAi), which uses a double-stranded RNA having a sequence identical or similar to the sequence of the target CPC-operon gene(s). RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. The introduced double-stranded RNA is initially cleaved into small fragments, which then serve as indexes of the target gene in some manner, thereby degrading the target gene. RNAi is known to be also effective in plants (see, e.g., Chuang, C. F. & Meyerowitz, E. M., Proc. Natl. Acad. Sci. USA 97: 4985 (2000); Waterhouse et al., Proc. Natl. Acad. Sci. USA 95:13959-13964 (1998); Tabara et al. Science 282:430-431 (1998)). For example, to achieve suppression of the expression of a DNA encoding a protein using RNAi, a double-stranded RNA having the sequence of a DNA encoding the protein, or a substantially similar sequence thereof (including those engineered not to translate the protein) or fragment thereof, is introduced into a plant of interest, e.g., green algae. The resulting plants may then be screened for a phenotype associated with the target protein and/or by monitoring steady-state RNA levels for transcripts encoding the protein. Although the genes used for RNAi need not be completely identical to the target gene, they may be at least 70%, 80%, 90%, 95% or more identical to the target gene sequence. See, e.g., U.S. Patent Application Publication No. 2004/0029283. Constructs encoding an RNA molecule with a stem-loop structure that is unrelated to the target gene and that is positioned distally to a sequence specific for the gene of interest may also be used to inhibit target gene expression. See, e.g., U.S. Patent Application Publication No. 2003/0221211.

The RNAi polynucleotides may encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 15, 20, 25, 30, 50, 100, 150, 200, or more nucleotides in length.

Expression vectors that continually express siRNA in transiently- and stably-transfected cells have been engineered to express small hairpin RNAs, which get processed in viva into siRNAs molecules capable of carrying out gene-specific silencing (Brummelkamp et al., Science 296:550-553 (2002), and Paddison, et al., Genes & Dev. 16:948-958 (2002)). Post-transcriptional gene silencing by double-stranded RNA is discussed in further detail by Hammond et al. Nature Rev Gen 2: 110-119 (2001), Fire et al. Nature 391: 806-811 (1998) and Timmons and Fire Nature 395: 854 (1998).

### Screening for Plants Having a Disrupted CPC Operon

The invention also provides methods of screening cyanobacteria having a disrupted CPC to select plants that are deficient in phycocyanin. Such plants can be generated using the techniques described above to target CPC-operon genes specifically. In other embodiments, mutagenized cyanobacteria e can be screened for a disruption in the CPC-operon.

Methods for introducing genetic mutations into cyanobacteria genes and selecting cyanobacteria with desired traits are well known. For instance, cells can be treated with a mutagenic chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. In other embodiments, insertional mutagenesis can be performed.

Cyanobacteria with mutations can be screened for a disruption to the CPC-operon. For example, decreased in the amount of a CPC-operon-encoded protein or mRNA may be analyzed, e.g., using techniques such as immunoassays, PCR and the like to detect cyanobacteria that have inhibited expression of one or more CPC-operon genes. Mutagenized cyanobacteria can also be evaluated by analyzing phycocyanin amounts and/or antenna size and selecting plants having a smaller, or truncated, antenna size relative to a wildtype cyanobacteria. In some embodiments, cyanobacteria are selected that have a 40% reduction, or greater, in antenna size relative in normal. In some embodiments, cyanobacteria are selected that have at least a 50%, or at least a 60% or 70% reduction in antenna size. In some embodiments, cyanobacteria are selected that have at least an 80% reduction in antenna size.

### Conditions for Culturing a Cyanobacteria Disrupted in the CPC-Operon to Increase Photosynthetic Productivity.

Cyanobacteria having a disruption in the CPC-operon as described herein that results in decreased phycobilisome light-harvesting antenna size have increased photosynthetic efficiency and productivity in mass cultures when grown under suitable conditions. These conditions are density and light intensity dependent. Thus, cyanobacteria cultured under conditions in which light intensity is at least 500 micromol photons per square meter per second, e.g., or at least 600, or at least 700 photons per square meter per second, and typically at least 800 micromol photons per square meter per second, and culture depth and cell density in the culture combine to result in more than 50% absorption of incoming light through the complete depth of a photobioreactor in which the culture is grown, or more than 60%, 65%, 70%, 75%, 80%, or 85% absorption of incoming light, and typically more than 90% or 95% absorption of incoming light, have increased photosynthetic efficiency and productivity. For example, for cyanobacteria cultured in a sunlight intensity of at least 800 micromol photons per square meter per second, 12 cm reactor depth with a cell density equivalent to 0.5 g dry cell weight is sufficient to absorb more than 90% of incoming sunlight. Similarly, a 6 cm depth at 1 g dry cell weight, or 24 cm depth at 0.25 g dry cell weight would satisfy the requirement of more than 90% absorption of incoming sunlight.

Transmittance of sunlight through a photobioreactor containing photosynthetic microorganisms can be measured using well-known techniques with a variety of instruments commercially available. For example, the Li-Cor Model LI-185B Quantum Radiometer/Photometer, when equipped with the right sensor, affords measurements of Photosynthetically Active Radiation (PAR, measured in the visible region of the spectrum). When the sensor is placed in the direction of the sun at the surface of the photobioreactor, it measures the intensity of incident sunlight. When immersed in the bottom of the photobioreactor it measures the intensity of the transmitted sunlight, after the latter has been filtered through by the absorbing photosynthetic microorganisms. Immersing the sensor in the photobioreactor at positions intermediate to the surface and bottom provides a measure of transmitted sunlight at variable distances from the surface of the reactor for instances when it is desirable to obtain measurements at distances less than the entire depth of the reactor.

Cyanobacteria can be used in high density photobioreactors (see, e.g., Lee et al., Biotech. Bioengineering 44:1161-1167, 1994; Chaumont, J Appl. Phycology 5:593-604, 1990), bioreactors for sewage and waste water treatments (e.g., Sawayama et al., Appl. Micro. Biotech., 41:729-731, 1994; Lincoln, Bulletin De L'institut Oceangraphique (Monaco), 12:109-115, 1993), elimination of heavy metals from contaminated water (e.g., Wilkinson, Biotech. Letters, 11:861-864, 1989), the production of β-carotene (e.g., Yamaoka, Seibutsu-Kogaku Kaishi, 72:111-114, 1994), the production of hydrogen (e.g., U.S. Patent Application Publication No. 20030162273), and pharmaceutical compounds (e.g., Cannell, 1990), as well as nutritional supplements for both humans and animals (Becker, 1993, “Bulletin De L'institut Oceanographique (Monaco), 12, 141-155) and for the production of other compounds of nutritional value.

Photosynthetic activity in cyanobacteria grown under the conditions described herein compared to counterpart cyanobacteria having a native CPC-operon can be evaluated using known assays, such as the illustrative assays in the examples section. For example, oxygen evolution activity of the cultures can be measured using an oxygen electrode illuminated with white actinic light from a quartz halogen lamp projector. Samples of cell suspension containing equal amounts of chlorophyll are loaded onto the oxygen electrode chamber. Sodium bicarbonate is added to the cell suspension prior to the oxygen evolution measurements to ensure that photosynthesis and oxygen evolution are not limited by the carbon supply available to the cells. After registration of the rate of dark respiration by the cells, samples are illuminated with gradually increasing light intensities. The rate of oxygen exchange (uptake or evolution) under each of these irradiance conditions is recorded continuously for 2-5 min allowing a linear regression of the slope. The light-saturation curve of photosynthesis is thus constructed, which plots the rate of photosynthesis as a function of incident irradiance. Cyanobacteria disrupted in the CPC-operon typically have about the same light-saturated rate as the wild type but have at least a 50%, or at least 100%, or 150% greater intensity for the half-saturation of photosynthesis. For example, the half-saturation intensity of photosynthesis for wild type and Δcpc transformant strains was measured to be 220 micromol photons m⁻² s⁻¹ for the wild type and 500 micromol photons m⁻² s⁻¹ for the Δcpc transformant. Those skilled in the art understand that an early light-saturation of wild type photosynthesis results in the wasteful loss of excess absorbed photons, whereas the later light-saturation of Δcpc photosynthesis alleviates the wasteful loss of photons, resulting in greater photosynthetic culture productivity. Photosynthetic productivity of a cyanobacterial mass culture grown under the conditions described herein compared to counterpart cyanobacteria having a native CPC-operon can be evaluated using known assays, such as the illustrative assays in the examples section (e.g., paragraph [0091]).

In typical embodiments, cyanobacteria culture can be diluted with fresh growth media at the end of the growth phase, e.g., at about 0.9-1.0 mg dcw mL⁻¹, to ensure that nutrient availability will not adversely affect growth, and to also permit for a continuous production process over a long growth period.

Cyanobacteria that are engineered to disrupt the CPC-operon in accordance with the invention may also be genetically modified with respect to other genes. For example, in some embodiments, the cyanobacteria may also comprise a heterologous isoprene synthase gene operably linked to a promoter (see, e.g., U.S. Pat. No. 7,947,478; WO 2008/003078) or to produce another product, e.g., that can be used to enhance production of ethanol or butanol.

### EXAMPLES

The examples described herein are provided by way of illustration only and not by way of limitation. Those of skill in the art will readily recognize a variety of non-critical parameters that could be changed or modified to yield essentially similar results.

Introductory Summary of Illustrative Results from the Experiments in the Examples

A phycocyanin-deletion mutant of Synechocystis (cyanobacteria) was generated upon replacement of the CPC-operon with a kanamycin resistance cassette. The Δcpc transformant strains (Δcpc) showed a green phenotype, compared to the blue green of the wild type (WT), lacked the distinct phycocyanin absorbance at 625 nm, had a lower Ch1 per cell content and a lower PSI/PSII reaction center ratio compared to the WT. Molecular and genetic analyses showed replacement of all WT copies of the Synechocystis DNA with the transgenic version. Biochemical analyses showed absence of the phycocyanin α- and β-subunits, of the corresponding linker polypeptides, and overexpression of the kanamycin resistance NPTI protein in the Δcpc. Physiological analyses revealed a higher, by a factor of about 2, intensity for the saturation of photosynthesis in the Δcpc compared to the WT. Under limiting intensities of illumination, growth of the Δcpc was slower than that of the WT. This discrepancy in the rate of cell duplication diminished gradually as growth irradiance increased. At about 800-1000 μmol photons m⁻² s⁻¹, the rate of cell duplication by the Δcpc was estimated to be the same as that for the WT. Culture productivity analyses under simulated bright sunlight and high cell-density conditions showed that biomass accumulation by the Δcpc was 1.57-times greater than that achieved by the WT. Thus, this example demonstrates the applicability of the Truncated Light-harvesting Antenna (TLA)-concept in cyanobacteria to provide substantial improvements in the photosynthetic efficiency and productivity of mass cultures upon minimizing the phycobilisome light-harvesting antenna size.

### Example 1. Construction of a Phycocyanin Deficient Synechocystis Strain

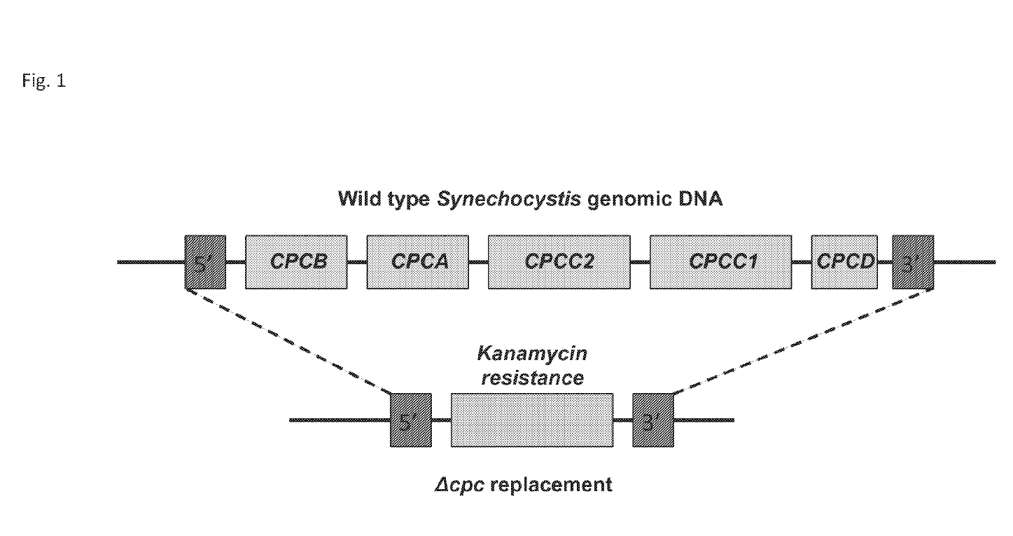

To investigate whether the concept of the Truncated Light-harvesting Antenna (TLA) of the photosystems, originally developed in green microalgae (Kirst and Melis 2014) can be applied to cyanobacteria, as a way by which to improve culture productivity under bright sunlight and high cell-density conditions, we generated and tested transformants with a severe reduction in the size of the phycobilisome (PBS) light-harvesting antenna. This was achieved upon deletion of the CPC DNA operon in Synechocystis, encoding for most of the proteins needed for the assembly of the PBS-peripheral phycocyanin rods. The CPC-operon, includes the CPCA gene, encoding for the phycocyanin α-subunit, the CPCB gene encoding the PC β-subunit, the CPCC1 and CPCC2 genes encoding phycocyanin rod linker polypeptides and the CPCD gene encoding a small linker polypeptide. The CPC operon was replaced via double homologous recombination with a neomycin phosphotransferase I (NPTI) gene, conferring kanamycin resistance, as a selectable marker, under the control of the endogenous CPC-operon promoter (FIG. 1).

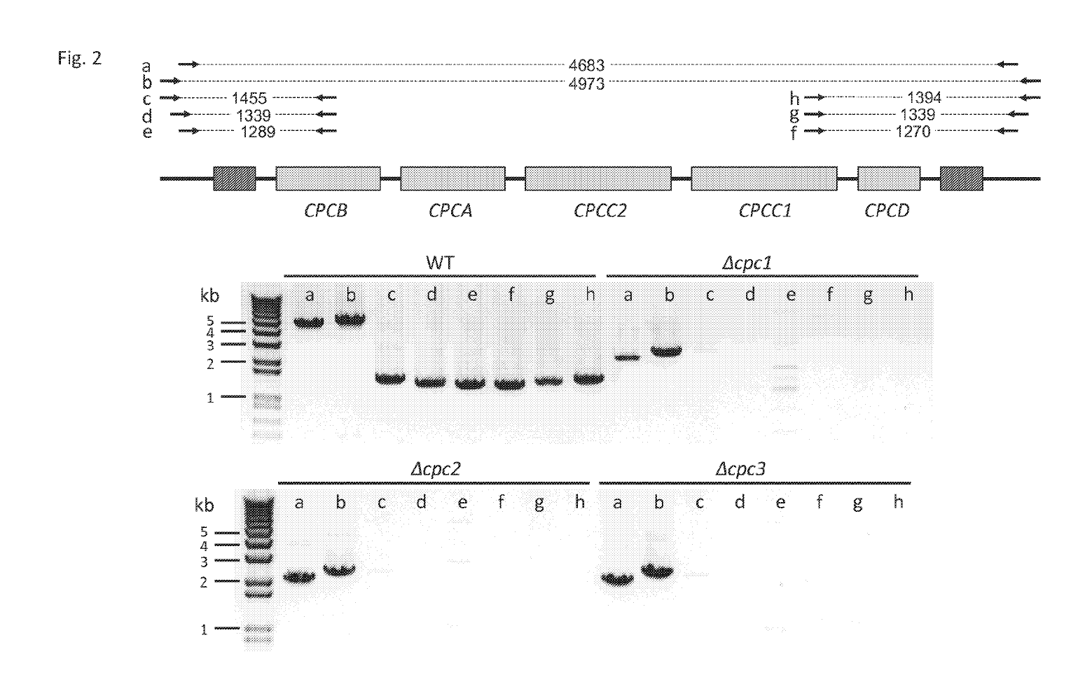

Genomic DNA PCR reactions were used to map the insertion site and to test for transformant DNA copy homoplasmy, i.e., to ensure that wild type copies of the DAN are deleted and that every copy of the resultant transformant cyanobacterial genome contains the NPTI gene and lacks the endogenous CPC-operon (FIG. 2). Primer set “a” in FIG. 2 tested for the correct integration of the insert at the CPC-operon site by using primers outside of the region of the homologous recombination, designed to amplify the entire CPC-operon and yielding different size fragments from the wild type and transformant DNA. In this case, the PCR product size using wild type (WT) genome as a template was a single 4,683 bp product (FIG. 2 WT a). Three independent Δcpc transformant lines generated a single product of 2,126 bp (FIG. 2, Δcpc1 a, Δcpc2 a, and Δcpc3 a) due to the replacement of the CPC-operon with the NPTI gene. The smaller product size observed for the transformants in FIG. 2 (Δcpc1 a, Δcpc2 a, and Δcpc3 a) indicated replacement of the CPC-operon with NPTI. It is also important to note that the Δcpc1, Δcpc2, and Δcpc3 transformants did not generate a 4,683 bp wild type product, suggesting that the transformants have achieved transgenic DNA copy homoplasmy, meaning that no copy of the wild type genome remains in the transformant cells.

Primer set “b” in FIG. 2 was used to show reproducibility of the above findings by a different set of primers and by amplifying a PCR product that is slightly longer than that in FIG. 2a, with expected sizes of 4,973 for the wild type and 2,416 bp for the Δcpc transformants, respectively. In this case, the PCR product size using WT genome as a template was a single 4,973 bp product (FIG. 2 WT b). The three independent Δcpc transformant lines generated a single product of 2,416 bp (FIG. 2, Δcpc1 b, Δcpc2 b, and Δcpc3 b) due to the replacement of the CPC-operon with the NPTI gene. Here again it was noted that the Δcpc1, Δcpc2, and Δcpc3 transformants did not generate a 4,973 bp wild type product, confirming that the transformants have achieved transgenic DNA copy homoplasmy.

Primer set “c”, “d”, “e”, “f”, “g” and “h” were all designed to yield PCR products specific to the wild type genomic DNA because one primer anneals to the native CPC-operon and thus, would not generate a product if the CPC-operon was quantitative replaced by NPTI in all Synechocystis DNA copies. These set of primers tested specifically for the presence of wild type genomic DNA copies in the transformants and, because of the sensitivity of a PCR reaction, this was designed to identify transformant lines that have not yet reached homoplasmy. The results of FIG. 2 (c, d, e, f, g, h) showed that PCR products were obtained with wild type DNA as the template of the PCR reaction and that none of the three Δcpc1, Δcpc2 and Δcpc3 independent transformant lines generated products using these primers. This is further evidence showing that Synechocystis Δcpc1, Δcpc2 and Δcpc3 transformant lines lacked wild type copies of DNA in their respective genomes.

### ΔCPC Transformant Phenotype Analysis

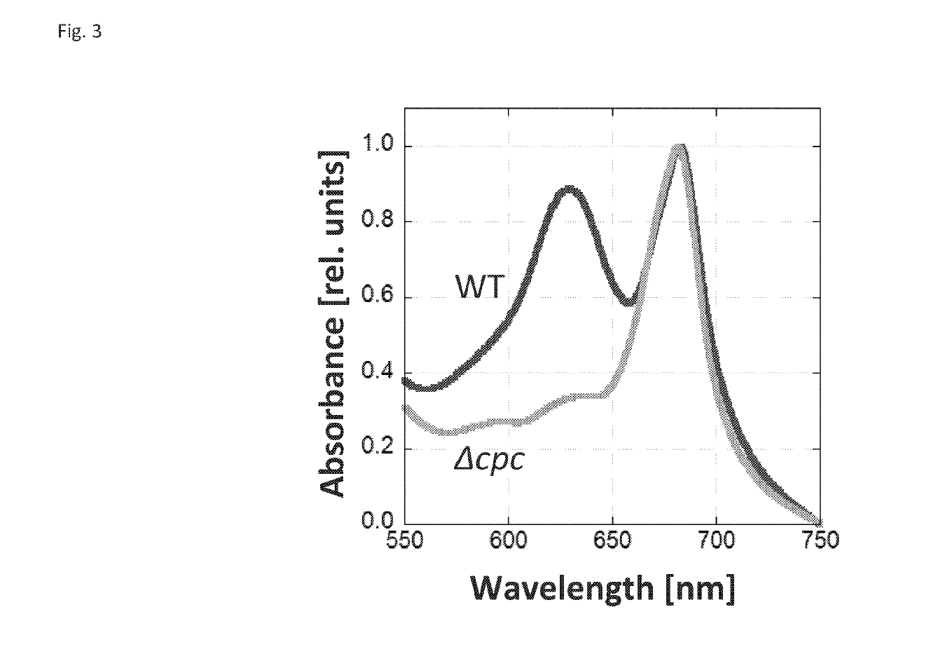

A change in the pigmentation of the Δcpc transformants was noted by the coloration of the colonies, which were lighter green compared to the blue-green coloration of the wild type. To investigate this pigmentation difference, we measured absorption spectra of live cells from wild type and Δcpc transformants (FIG. 3). The absorption spectrum of the cells of the wild type showed the typical absorbance bands of chlorophyll at 680 nm and phycocyanin at 625 nm (Glazer and Hixon, 1975; Glazer 1989). The Δcpc transformants showed the specific Ch1 absorption peak at 680 nm, whereas the phycocyanin absorption peak at around 625 nm was missing. This is attributed to the deletion of the CPC-operon and the resulting absence of phycocyanin from the Δcpc transformants.

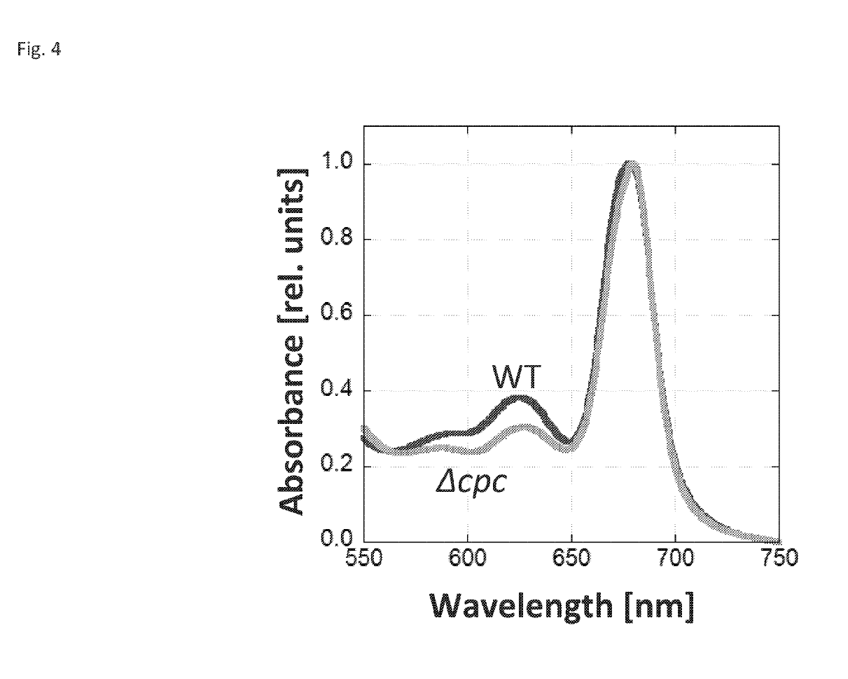

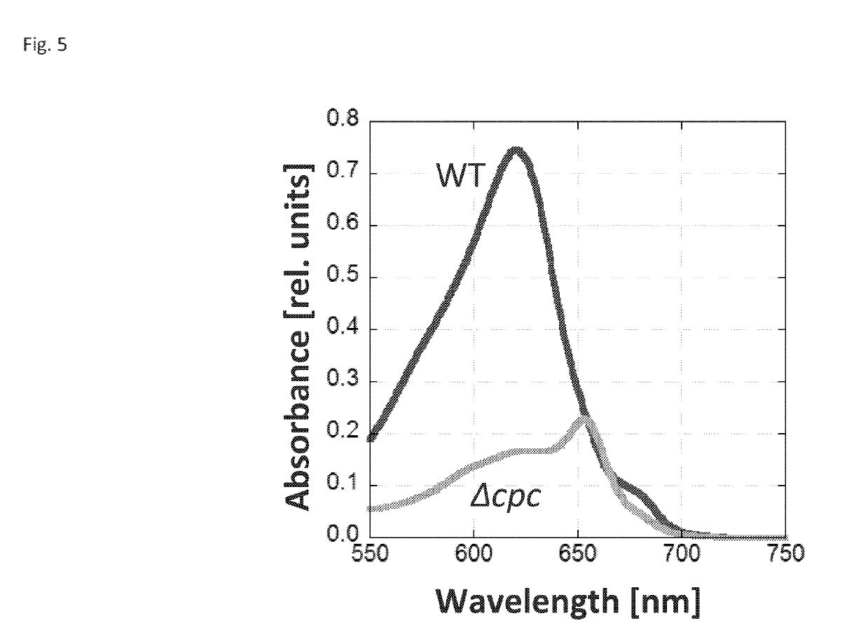

Cell lysates were fractionated into crude membrane and soluble fractions. The membrane fraction of wild type and Δcpc transformants (FIG. 4) showed similar absorption spectra in the red region, dominated by the absorption of Ch1 at 680 nm. This was expected, because Ch1 pigments are bound to the transmembrane proteins of the PSI-core and PSII-core complexes, which pelleted with the thylakoid membranes upon centrifugation. The soluble fraction of wild type and Δcpc transformants contained the dissociated phycobilisomes (FIG. 5), and showed substantially different absorption spectra. The wild type supernatant was blue and dominated by the absorption of phycocyanin at 625 nm, with minor absorbance shoulders at 650 and 675 nm, whereas that of the Δcpc transformants showed a featureless low-level absorption in the 625 nm region, a peak at 650 nm, and a minor band at 675 nm. The latter are ascribed to allophycocyanin (Glazer 1989), which remains in the residual phycobilisome as a component of the core-cylinders of the Δcpc transformants. Missing from the latter was the dominant 625 nm phycocyanin absorbance.

The chlorophyll content of the cells, measured on per OD₇₅₀ or dry-cell-weight (dcw) basis in the Δcpc transformants was 60-70% of that of the wild type, while the carotenoid content did not change significantly (Table 2). Changes in Ch1 content may reflect underlining changes in photosystem stoichiometry, as a result of the Δcpc transformations. Past work with TLA mutants showed that the PSI/PSII stoichiometry ratio was adjusted and optimized when mutations induced light-harvesting antenna size changes that affected the two photosystems in a dissimilar manner (Melis 1991). In this respect, PSI is more abundant than PSII in cyanobacteria and contains 95 Ch1 a and 22 β-carotene molecules, whereas the PSII-core contains only 37 Ch1 a and 11 β-carotene molecules (Glick and Melis 1988; Jordan et al. 2001; Umena et al. 2011). The sizable PBS antenna aids absorption of sunlight by PSII, so that a statistically balanced distribution of excitation between the two photosystems is achieved with a PSI/PSII ratio of about 2:1 to 4:1 in the wild type (Melis and Brown 1980; Myers et al. 1980; Fujita et al. 1987; Glazer and Melis 1987). Deletion of the peripheral phycocyanin rods from the Δcpc transformants lowers the capacity and rates of light absorption by PSII and, thus, would tend to tilt the balance of excitation energy distribution in favor of PSI. A decline in the number of the PSI units in the Δcpc transformants would then be a compensation response, entailing adjustment and optimization of function (Melis 1991), thereby explaining the lower Ch1 per cell in these transformants (Table 1).

To test the hypothesis of an adjusted and optimized photosystem stoichiometry in the Δcpc transformants, and to investigate the functional effect of the truncation of the PBS-PSII antenna size more precisely, we applied light-induced absorbance difference spectrophotometry to quantify PSI and PSII reaction centers and, thus, to estimate the PSI/PSII ratio in wild type and Δcpc transformants (Melis 1989). For the PSI measurement, the amplitude of the light-induced ΔA₇₀₀ signal measured the amount of P700 in the sample. For the PSII measurement, the amplitude of the light-induced ΔA₃₂₀ signal measured the amount of Q_{A} (Melis 1989). Ratios of P700 per total Ch1 content and Q_{A}/Ch1 in Synechocystis thylakoid membranes are shown in Table 2, as is the resulting PSI/PSII stoichiometric ratio. The PSI/PSII ratio declined from about 2.5:1 in the wild type down to 1.8:1 in the Δcpc transformants. This adjustment is consistent with the hypothesis of an optimized PSI/PSI I ratio in the Δcpc transformants in the direction of balancing the excitation energy distribution between the two photosystems (Glazer and Melis 1987).

The effective absorption cross-section of PSI and PSII were compared in the wild type and Δcpc transformants. This was measured from the light-induced ΔA₇₀₀ oxidation kinetics of P700 for PSI and the Q_{A} fluorescence induction kinetics for PSII, measured under weak broad-band green actinic excitation defined by CS 3-69 and CS 4-96 Corning filters (half-band width of 40 nm with 50% transmittance at 520 and 560 nm, respectively). Such green excitation would sensitize phycocyanin much more than it would sensitize allophycocyanin and chlorophyll. In wild type live cells, rates of light absorption by PSII (k_{PSII}, Table 2) were 30.1 s⁻¹, whereas in the Δcpc transformant lines they averaged 3.7 s⁻¹. The substantial, almost 10-fold difference in the effective absorption cross-section of PSII in wild type and Δcpc transformant cells is attributed to the presence and absence of phycocyanin between the two respective strains. Rates of light absorption by PSI (k_{PSI}, Table 2) were determined with isolated thylakoid membranes from which the phycobilisome peripheral antenna was disconnected. As such, the results afford a direct comparison of the core Ch1 antenna size of PSI between wild type and Δcpc transformants. Similar rate constants of P700 photo-oxidation with values between 2.9 and 2.5 s⁻¹ were measured (k_{PSI}, Table 2), suggesting similar PSI Ch1 antennae. This finding is consistent with the notion that photosystem stoichiometry adjustments affect the ratio of the two photosystems but not necessarily their core Ch1 antenna size.

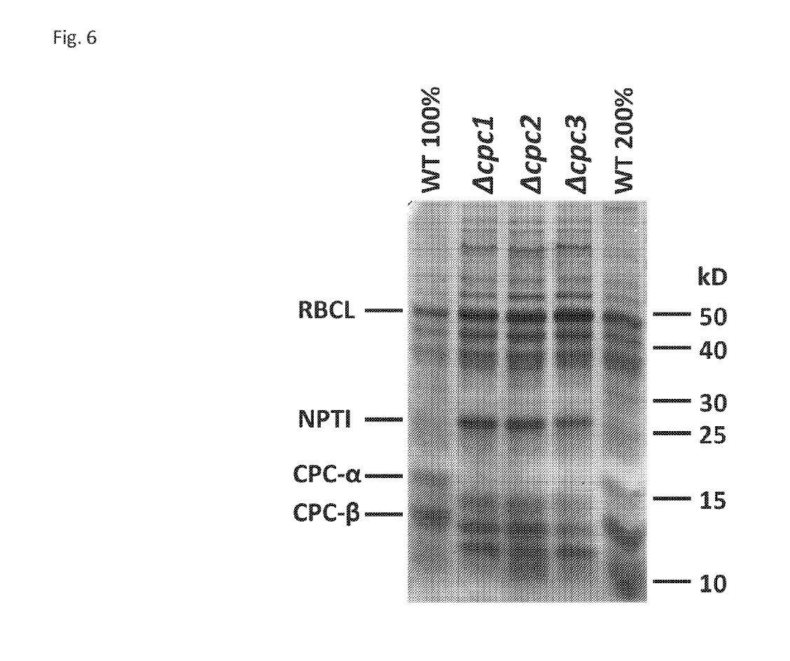

SDS-PAGE and Western blot analysis of total protein extracts from wild type and Δcpc transformants provided further insight into the phenotype of the latter (FIG. 6). In the wild type, the phycocyanin alpha and beta subunits were visible as abundant low molecular weight proteins (FIG. 6, CPC-α and CPC-β). These protein bands were absent from the Δcpc transformants. The latter showed a new pronounced band at around 27 kD, which was identified by Western blot analysis to be the NPTI protein (not shown), conferring the kanamycin resistance to the Δcpc transformants. Absence of the phycocyanin alpha and beta subunits from the Δcpc transformants is consistent with the absorbance spectral measurements, and PSII absorption cross section results, and is further consistent with the notion of a severely truncated phycobilisome antenna size in the Δcpc transformants, one that contains only the core allophycocyanin component, as the auxiliary antenna of the PSII reaction center.

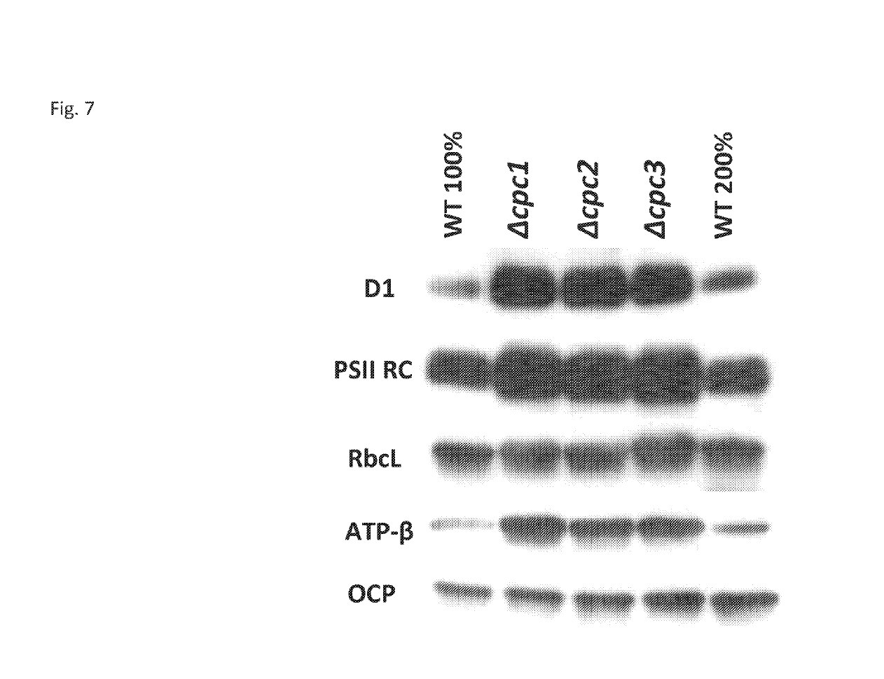

Western blot analyses of protein extracts from wild type and Δcpc transformants, probed with specific polyclonal antibodies raised against the D1 protein, the PSII reaction center D1/D2 heterodimer (PSII RC), the RbcL, ATP synthase β-subunit (ATP-β), and the orange carotenoid protein (OCP) showed presence of these reference proteins in wild type and Δcpc transformants (FIG. 7). Loaded on a per Ch1 basis, three lines of the Δcpc transformants showed greater relative amount of these proteins, as compared to the wild type. Δcpc transformants have only about 60% of the Ch1/cell content and a greater PSII/PSI ratio. Loading on a per Ch1 basis would tend to overload the Δcpc transformant lanes for PSII compared to that of the wild type (Table 2, D1 and PSII RC). A similar argument can be made for the ATP-β subunit (Table 2, ATP-β). On the other hand, non-thylakoid membrane proteins such as the RbcL and OCP occur in about equivalent amounts in wild type and Δcpc transformants.

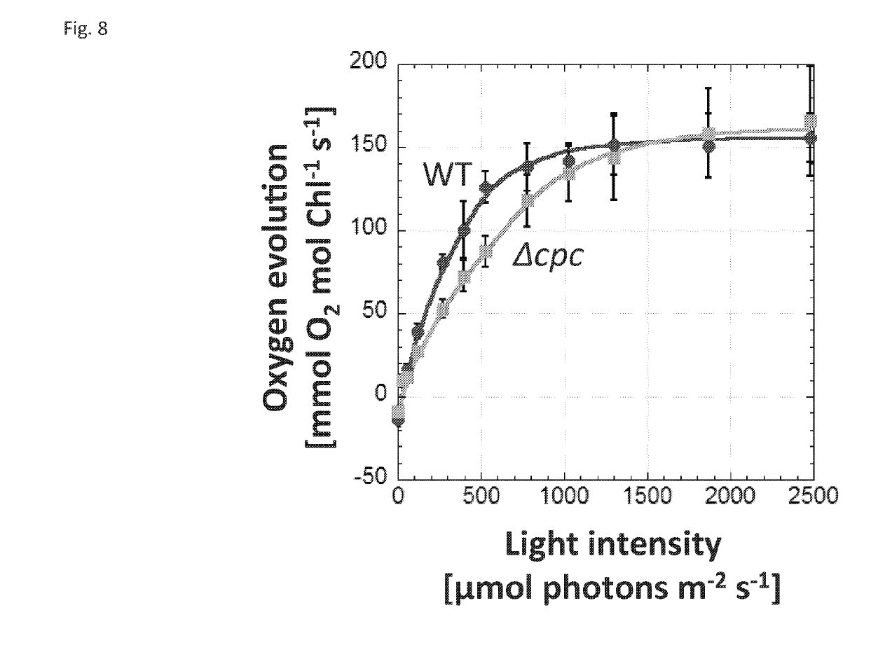

The functional consequence of the deletion of phycocyanin from the phycobilisome antenna in the Δcpc transformants was assessed upon measurement of the light saturation curves of photosynthesis in the strains. These are shown in FIG. 8 for wild type and Δcpc transformants, normalized to the Ch1 concentration of the samples. At zero light intensity, the oxygen evolution rate was negative, reflecting the respiratory activity of the cells. It was measured to be on the average 13±4 and 9±3 mmol O₂ per mol Ch1 per s for the wild type and Δcpc transformants, respectively. The rate of photosynthesis increased linearly as a function of light-intensity in the range between 0-300 μmol photons m⁻² s (FIG. 8). The slope of this line was steeper for the wild type than for the Δcpc transformants because of the presence of phycocyanin in the wild type, which affords greater absorption and utilization of actinic light than that in the Δcpc transformants. This was also reflected in the half-saturation intensity of photosynthesis for the two strains, which was measured to be 220 μmol photons m⁻² s⁻¹ for the wild type and 500 μmol photons m⁻² s⁻¹ for the Δcpc transformants. This difference is consistent with the TLA concept, i.e., the effect of antenna truncation on the light saturation curves of photosynthesis, as previously measured in green microalgae (Kirst et al. 2012a; 2012b). The light-saturated rate of photosynthesis, when measured on a chlorophyll basis (FIG. 8) was slightly greater for the Δcpc transformants and the wild type, reflecting changes in cellular chlorophyll content due to the photosystem stoichiometry adjustment.

The TLA concept and the two-fold greater intensity needed to half-saturate the rate of photosynthesis in the Δcpc transformants compared to the wild type predicts greater productivity for cultures of the transformants under high cell density and saturating illumination (greater than 1,000 μmol photons m⁻² s⁻¹) conditions, because fewer photons would be wasted via non-photochemical quenching processes in the Δcpc transformants (Melis 2009; Melis 2012).

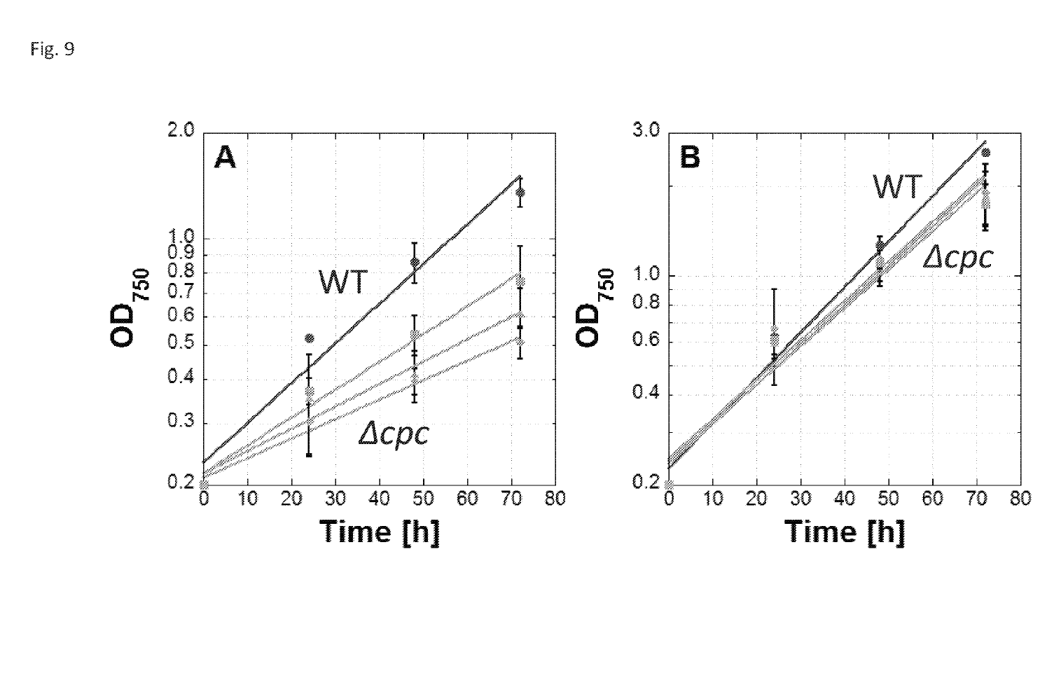

The saturation intensity of photosynthesis (Is) for wild type and Δcpc transformants was defined from the intercept between the initial linear increase in the rate of photosynthesis as a function of light intensity and the asymptotic light-saturated rate achieved at maximum intensity, e.g. 2,500 μmol photons m⁻² s⁻¹. This Is was calculated to be 400 μmol photons m⁻² s⁻¹ for the wild type and 1,000 μmol photons m⁻² s⁻¹ for the Δcpc transformants. Again, in cultures under high cell density conditions, the two-fold greater intensity for the saturation of photosynthesis in the Δcpc transformants compared to the wild type informs that there would be a quantitative productivity difference between the two strains under saturating illumination. This, however, will be manifested at light intensities greater than 1,000 μmol photons m⁻² s⁻¹, i.e., at light intensities greater than what is needed for the saturation of photosynthesis in the Δcpc transformants. To investigate the photosynthetic productivity of wild type and Δcpc cultures in greater detail, we measured rates of cell growth and biomass accumulation at different light intensities ranging from sub-saturating to saturating. At the low light intensity of 50 μmol photons m⁻² s⁻¹, Δcpc transformants showed growth slower than that of the wild type (part A of FIG. 9). The doubling time under these conditions was measured to be 30 h for the wild type and about 49 h for the Δcpc transformants. This retarded growth rate under low light intensities is a consequence of the severe light-limitation imposed on the Δcpc transformants due to the absence of phycocyanin, whereby the latter do not harvest as much light energy as the wild type and, therefore, photosynthesis and growth is limited in these mutants. This discrepancy in the growth phenotype became less severe, when the cell growth intensity was increased. When grown under 170 μmol photons m⁻² s⁻¹, the difference in growth rates between wild type and the Δcpc transformants was diminished (part B of FIG. 9) with doubling times of 20.5 h for the wild type and 26.9 h for the Δcpc transformants. This finding is consistent with recent studies (Page et al. 2012; Liberton et al. 2013), where phycocyanin-less mutants showed a retarded growth under low light intensities between 50 and 150 μmol photons m⁻² s⁻¹. However, when growth was measured under 350 mol photons m⁻² s⁻¹ this difference between wild type and Δcpc transformants was minimized with doubling times of 16.7 h and 19.5 h for the wild type and Δcpc transformants, respectively.

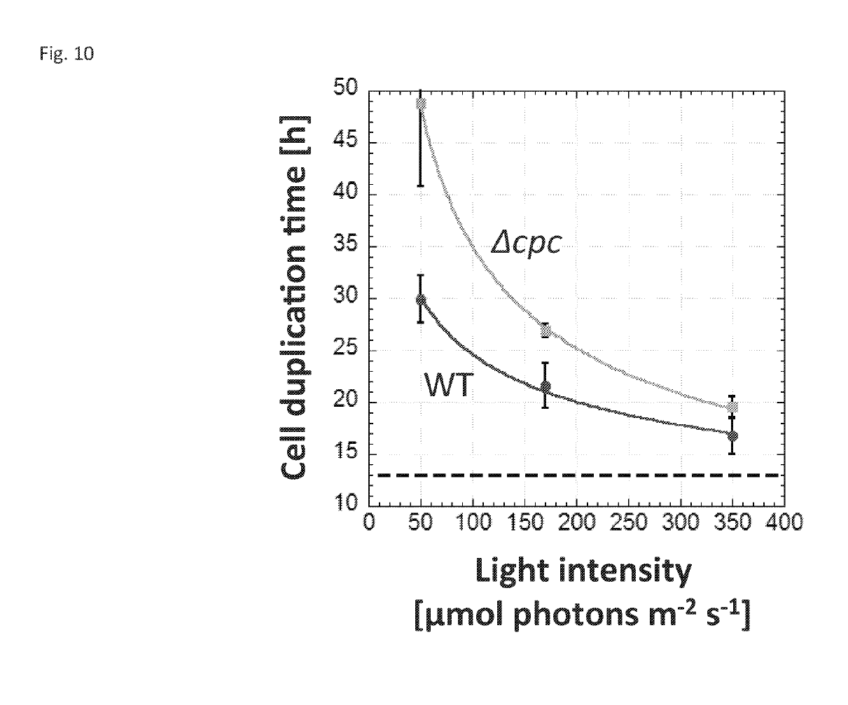

A more extensive presentation of the measured cell duplication time in Synechocystis cultures, as a function of growth light intensity, for wild type and the Δcpc transformants, is shown in FIG. 10. A substantial difference exists between cell duplication time in wild type and Δcpc transformants at low growth intensities. This difference is diminished as the growth intensity increases. Extrapolating the relationship to higher light intensities, we found that wild type and Δcpc transformants would reach the same cell duplication time of about 13 h at about 800-1000 μmol photons m⁻² s⁻¹ (dashed line in FIG. 10). It is evident from this analysis that wild type cells would have a competitive advantage when growth is measured under low light-intensities, but when the light-intensity approaches or exceeds the saturation point of photosynthesis, wild type and Δcpc transformants can grow with identical rates.

### Example 2. Culture Productivity Under High Cell Density and Saturating Irradiance Conditions

The TLA technology concept is based on the premise of maximizing sunlight utilization efficiency and photosynthetic productivity in mass microalgal cultures, or high-density plant canopies, upon minimizing the light-harvesting capacity of the photosynthetic apparatus (Melis 2009). The rationale for this counterintuitive concept is that, at saturating or greater intensities, a small light-harvesting antenna would alleviate excessive absorption of sunlight by the top layer of cells in a mass culture or canopy and would thus prevent the ensuing wasteful dissipation of the excess absorbed energy, while at the same time permitting light penetration deeper into the culture (Melis 2009; Melis 2012; Kirst and Melis 2014). It is worth noting that improved photosynthetic productivity of a culture with TLA cells is solely due to the effect of better sunlight penetration with more cells deeper into a culture having a chance to absorb and perform useful photosynthesis, whereas the photosynthetic productivity of individual TLA cells would not be better than that of the wild type. Thus, it is important in such applications that bioreactors are designed to be deep enough with a cell-density sufficient to permit quantitative absorption of all incoming irradiance, and to perform the productivity measurement at light intensities equal to or greater of that needed to saturate photosynthesis.

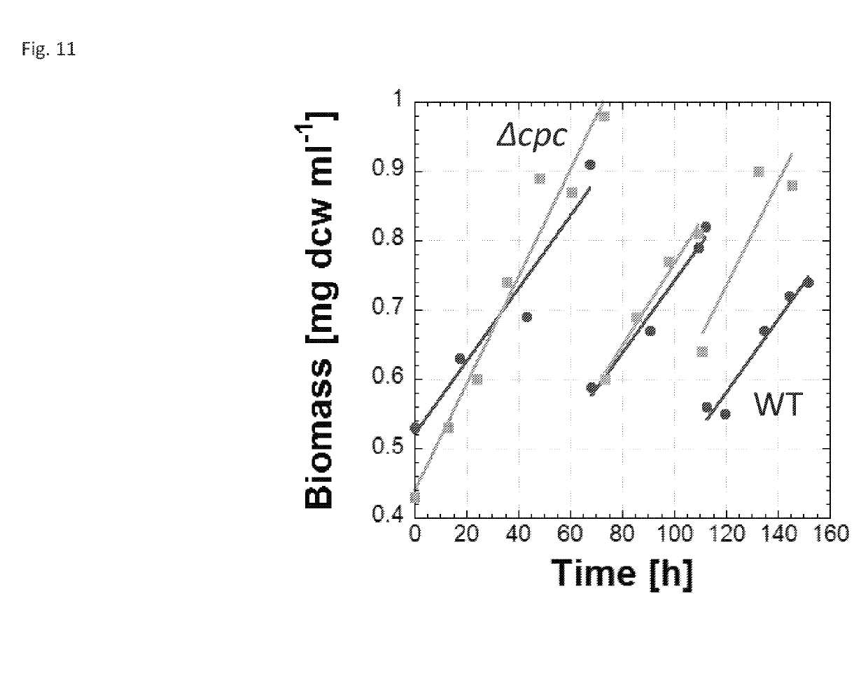

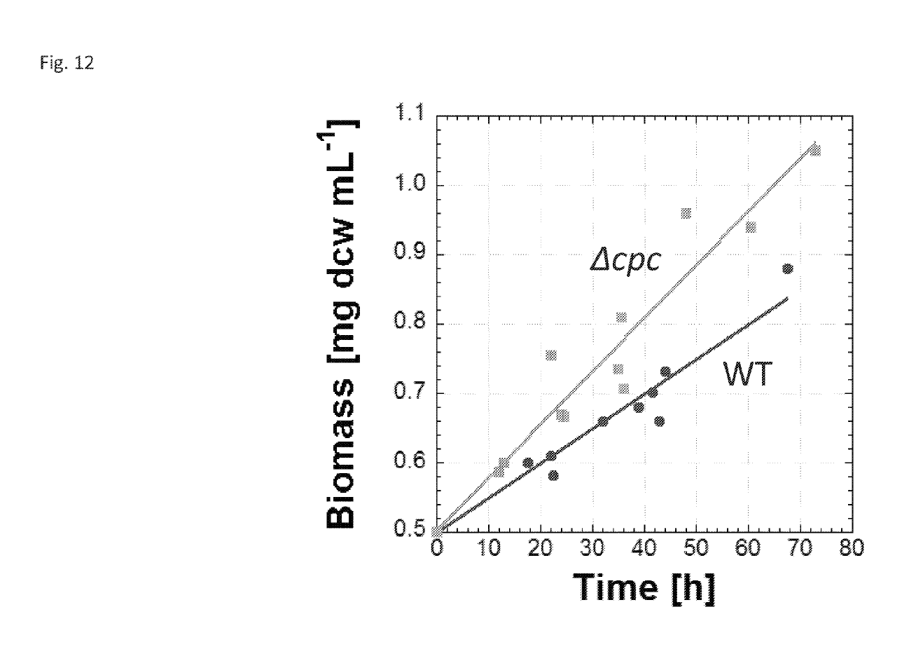

The light-saturation curves of photosynthesis (FIG. 8) indicated that Δcpc transformants are promising in the application of the TLA technology concept. This was tested upon wild type and Δcpc transformants growth under simulated bright sunlight conditions (e.g. 2,000 μmol photons m⁻² s⁻¹) in the laboratory, with cultures having optical density sufficient to absorb >98% of the incident irradiance. These conditions ensured that light-energy input for the wild type and Δcpc transformant cultures was about the same. Biomass accumulation results of representative wild type and Δcpc transformant cultures are shown in FIG. 11. In this experiment, cultures were diluted with fresh growth media once they approached the end of the growth phase, i.e., at about 0.9-1.0 mg dcw mL⁻¹, to ensure that nutrient availability will not adversely affect growth, and to also permit for a continuous production process over a long growth period, during which to assess the effect of the TLA phenotype on the productivity of the culture. The slope of the linear regressions in FIG. 12 showed the rate of biomass accumulation by wild type (FIG. 11, circles) and Δcpc transformant cultures (FIG. 11, squares), respectively. It is evident from the results that rates of biomass accumulation by the Δcpc transformant cultures were always faster than those by the wild type over all cultures and Δcpc transformant lines measured. We compiled results from several such continuous growth experiments for wild type and Δcpc transformant cultures (FIG. 12). In this presentation, initial cell density of the cultures was about 0.5 mg dcw mL⁻¹. The average rate of biomass accumulation in this presentation (FIG. 12) was defined by the slopes of the linear regression of the points and was measured to be about 4.9 μg dcw mL⁻¹ h⁻¹ for the wild type and 7.7 μg dcw mL⁻¹ h⁻¹ for the Δcpc transformants. This analysis, therefore, showed that culture productivity of the Δcpc transformants exceeded that of the wild type by about 57%.

Discussion of Illustrative Data from Examples 1 and 2.

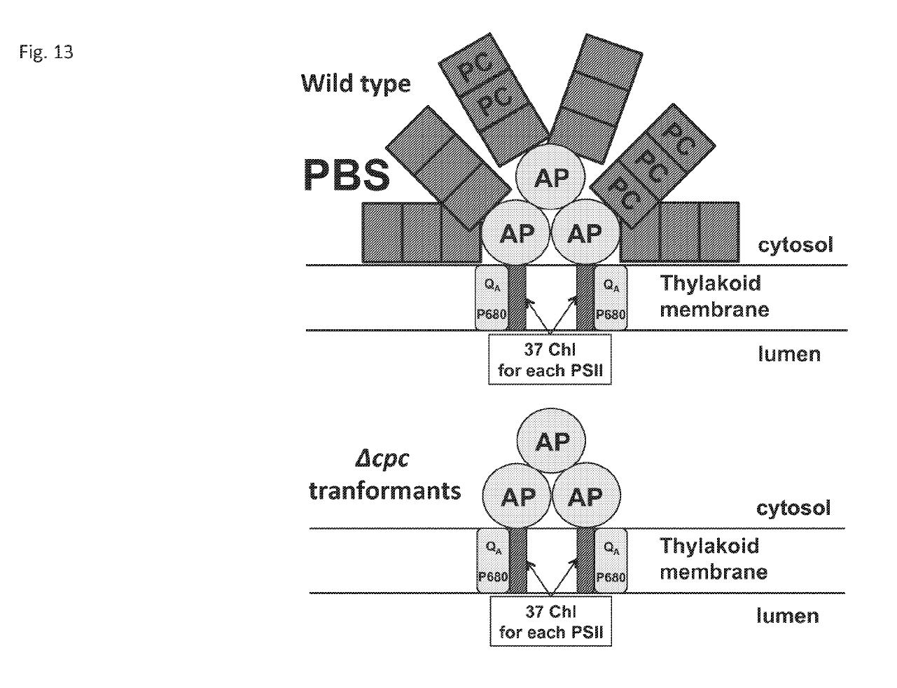

Deletion of the key cyanobacterial CPC-operon, encoding phycocyanin and associate linker polypeptides of the phycobilisome peripheral rods, caused a highly truncated phycobilisome antenna size, resulting in a substantially smaller absorption cross-section for PSII in the Synechocystis Δcpc transformants. A schematic model presentation of the phycobilisome structure and its association with photosystem II is shown in FIG. 13 for the wild type and the Δcpc Synechocystis transformants. Noted is the presence of the allophycocyanin (AP) core cylinders in both strains, and the absence of phycocyanin (PC) from the Δcpc transformants. A compensation reaction of the cells to the smaller PBS-PSII antenna size was a reduction in the number of PSI units, relative to those of PSII (Table 2), which can be viewed as a cellular effort to retain balanced absorption and distribution of excitation energy between the two photosystems (Glazer and Melis 1987). Such adjustment enables the Δcpc transformants to operate the linear electron transport process efficiently, compared to an unbalanced Δcpc transformants system in which the PSI/PSII ratio would be the same as that in the wild type.

The smaller phycobilisome antenna size in the Δcpc transformants did not confer growth advantage under low-light conditions.—This was previously reported by Nakajima and Ueada (1997; 1999), Bernat et al. (2009), Kwon et al. (2013). Joseph et al. (2014), as well as by Page et al. (2012). At light intensities greater than those required to saturate photosynthesis, e.g., greater than 800 micromol photons per square meter per second, Δcpc cultures as a whole, having a high-density of cells in a photobioreactor under direct sunlight would outperform a corresponding wild type culture as they would not over-absorb sunlight and wastefully dissipate the excess excitation energy. It was pointed out before with microalgal cultures that such TLA property could translate into a greater sunlight-to-biomass energy conversion efficiency of photosynthesis, helping to elevate the culture performance and productivity beyond what can be achieved with wild type strains (Melis 2009; Melis 2012; Kirst and Melis 2014). Results in this work provide evidence of a 57% improvement in the productivity of high cell density Δcpc transformant cultures as compared to that of the wild type, consistent with the predictions of the TLA model.

Considering the recent interest in the field of renewable biomass, and fuel and chemicals production by photosynthetic microorganisms (Melis 2007; Hankamer et al. 2007; Hu et al. 2008; Greenwell et al. 2010; Mata et al. 2010; Mayfield et al. 2007; Dauvillee et al. 2010; Michelet et al. 2011; Bernat et al. 2009), improvements in the energy conversion efficiency of photosynthesis can significantly improve the economic outlook of such processes using cyanobacteria or microalgae as a single-celled photocatalyst (Lindberg et al. 2010; Stephens et al. 2010; Bentley and Melis 2012).

Although +57% improvement in productivity was demonstrated with the Δcpc transformant cultures in example 2, improvements in photosynthetic energy conversion efficiency can be as high as 3-fold over that in the wild type (Melis 2009; Kirst and Melis 2014). Reasons for the relatively low yield of photosynthesis in the Δcpc transformants analyzed in these examples, could be attributed to the over-expression of the NPTI protein. A significant portion of carbon commitment by the cells goes into the synthesis of the NPTI protein in the Δcpc transformants as evidenced by the Coomassie-stained gel (FIG. 6), which protein has no useful function, whenever cells are gown in liquid media in the absence of kanamycin, as the case was in this work. If this carbon was invested into cell constituent proteins that contributed to growth, a faster rate of growth could potentially be achieved. Another concern and consideration is whether heterologous accumulation of such substantial amounts of the NPTI protein may affect fitness of the transformant lines through NPTI protein toxic effects or feedback inhibition. Further manipulation of the light-absorption properties of cyanobacteria, e.g. upon deletion of the allophycocyanin phycobilisome subunits, could also provide additional improvements in the sunlight-to-biomass energy conversion efficiency (Joseph et al. 2014).

Page et al. (2012) recently investigated the photosynthetic energy conversion efficiency of a similar phycobilisome-deficient mutant of Synechocystis lacking the phycocyanin peripheral antenna. Under low and medium light conditions, the phycocyanin-deletion mutants lagged in growth and productivity when compared to the wild type, as also reported in this work. These results led Page et al. (2012) and Liberton et al. (2013) to the generalized conclusion of a lowering in photoautotrophic productivity in the cyanobacterium Synechocystis by phycobilisome antenna truncation, opposite to the conclusions drawn by Nakajima and Ueda (1997; 1999), and also opposite to the conclusions described here. An explanation of the opposite conclusions could be that productivity of the phycocyanin-deletion mutants by Page et al. (2012) and Liberton et al. (2013) was not properly assessed at high cell-densities and light intensities equal to or above that required for the saturation of photosynthesis, when the TLA property ought to manifest in the form of improved biomass productivity. A high cell density culture, sufficient to absorb all incoming photosynthetically active radiation, and light intensities equal to or above those required for the saturation of photosynthesis are important requirements for the detection of improvements in the photosynthetic productivity of TLA versus wild type strains.

All publications, accession numbers, and patent applications cited in this specification are herein incorporated by reference as if each individual publication or patent application were specifically and individually indicated to be incorporated by reference.

TABLE 1
Primers used in the genomic DNA analysis
of Synechocystis wild type and Δcpc
transformants (SEQ ID NOS: 7-14, consecutively).
Primer sets refer to FIG. 2.

| | Used in | |
|---|---|---|
| Primer | primer set, | |
| Direction | see FIG. 2 | Sequence, 5′ to 3′ |
| Forward | b, c | GACTTGAATGTCACTAAC |
| | | TACATCCAGTCTTTGC |
| Forward | d | GCTAAATCCCATGAAGAG |
| | | AAGGTTTATG |
| Forward | a, e | CCATTAGCAAGGCAAATC |
| | | AAAGAC |
| Reverse | c, d, e | GGTGGAAACGGCTTCAGT |
| | | TAAAG |
| Forward | f, g, h | GTTCCCTTTGGTCAAGCA |
| | | AGTAAG |
| Reverse | a, f | GGTTGATTCGTTTACATC |
| | | AGTTCAATAAAG |
| Reverse | g | CCATTAAACATTGTGCTT |
| | | ACACTCC |
| Reverse | b, h | GAAGTGCCAGTGACTAAC |
| | | CTTTATCGAG |

TABLE 2
Pigment content, photosystem reaction center quantification,
and relative absorption cross-section of PSII and PSI in Synechocystis
wild type and Δcpc transformants grown photoautotrophically
in the laboratory. Photosystem absorption cross-sections and
reaction center concentrations were measured spectrophotometrically
(Melis 1989). n = 3; means ± SD.

| | | Average |
|---|---|---|
| Parameter measured | Wild Type | (Δcpc1, Δcpc2, Δcpc3) |
| Chl/OD₇₅₀ [μg] | 2.6 ± 0.1 | 1.8 ± 0.1 |
| Chl/dcw [μg · mg⁻¹] | 8.5 ± 0.7 | 5.0 ± 0.5 |
| Car/OD₇₅₀ [μg] | 2.2 ± 0.1 | 2.1 ± 0.2 |
| Car/dcw [μg · mg⁻¹] | 7.2 ± 0.7 | 6.5 ± 0.1 |
| Chl/Car | 1.2 ± 0.1 | 0.7 ± 0.1 |
| P700/Chl · 10⁻³ | 5.60 ± 0.34 | 5.82 ± 0.68 |
| Q_{A}/Chl · 10⁻³ | 2.23 ± 0.11 | 3.25 ± 0.18 |
| PSI/PSII | 2.5 ± 0.08 | 1.8 ± 0.13 |
| k_{PSII} (Q_{A} photoreduction | 30.1 ± 6.2 | 3.7 ± 1.5 |
| kinetics) [s⁻¹] | | |
| k_{PSI} (P700 photooxidation | 2.9 ± 0.2 | 2.5 ± 0.1 |
| kinetics) [s⁻¹] | | |

TABLE 3
Accession numbers for CPC genes and proteins
from illustrative cyanobacteria species.

| | | Protein accession | Operon/Genome |
|---|---|---|---|
| Species | Gene | number | accession number |
| Synechocystis sp. PCC 6803 | CpcB | NP_440552.1 | Total genomic DNA |
| Synechocystis sp. PCC 6803 | CpcA | NP_440551.1 | sequence: |
| Synechocystis sp. PCC 6803 | CpcC2 | NP_440550.1 | NC_000911.1 |
| Synechocystis sp. PCC 6803 | CpcC1 | NP_440549.1 | |
| Synechocystis sp. PCC 6803 | CpcD | NP_440548.1 | |
| Cyanothece sp. PCC 8801 | CpcB | YP_002373211.1 | Total genomic DNA |
| Cyanothece sp. PCC 8801 | CpcA | YP_002373212.1 | sequence: |
| Cyanothece sp. PCC 8801 | CpcC2 | YP_002373213.1 | NC_011726.1 |
| Cyanothece sp. PCC 8801 | CpcC1 | YP_002373214.1 | |
| Cyanothece sp. PCC 8801 | CpcD | YP_002373215.1 | |
| Synechococcus elongatus sp. PCC 6301 | CpcB | YP_171206.1 | Total genomic DNA |
| Synechococcus elongatus sp. PCC 6301 | CpcA | YP_171205.1 | sequence: |
| Synechococcus elongatus sp. PCC 6301 | CpcC2 | YP_171209.1 | AP008231.1 |
| Synechococcus elongatus sp. PCC 6301 | CpcC1 | YP_171208.1 | |
| Synechococcus elongatus sp. PCC 6301 | CpcD | YP_171207.1 | |
| Synechococcus sp. PCC 7002 | CpcB | YP_001735445.1 | Total genomic DNA |
| Synechococcus sp. PCC 7002 | CpcA | YP_001735446.1 | sequence: |
| Synechococcus sp. PCC 7002 | CpcC | YP_001735447.1 | NC_010475.1 |
| Synechococcus sp. PCC 7002 | CpcD | YP_001735448.1 | |
| Thermosynechococcus elongates BP-1 | CpcB | NP_682747.1 | Total genomic DNA |
| Thermosynechococcus elongates BP-1 | CpcA | NP_682748.1 | sequence: |
| Thermosynechococcus elongates BP-1 | CpcC | NP_682749.1 | NC_004113.1 |
| Thermosynechococcus elongates BP-1 | CpcD | NP_682750.1 | |

### Methodology for Examples 1 and 2

### Cell Cultivation

Synechocystis sp. PCC 6803 was used as the recipient strain, and is referred to as the wild type. Wild type and transformant strains were maintained on solid BG-11 media supplemented with 10 mM TES-NaOH (pH 8.2), 0.3% sodium thiosulfate at 25° C. and about 50 μmol photons m⁻² s⁻¹. When indicated, kanamycin was added to a concentration of 50 μg/mL. Liquid cultures were grown in 25 mM phosphate buffered BG11, pH 7.5, at 25.5° C. under constant aeration and were gradually acclimated to the final light intensity. Acclimation times were 3 d at 170 μmol photons m⁻² s⁻¹, 5 d at 350 μmol photons m⁻² s⁻¹, 14 d with a step-wise increase in the light intensity to 1,500 μmol photons m⁻² s⁻¹, and 20 d with a step-wise increase in the light intensity to 2,000 μmol photons m⁻² s⁻¹. Cultures grown under 2,000 μmol photons m⁻² s⁻¹ were bubbled continuously with 3% CO₂ to ensure that C-availability would not limit the rate of growth.

### Nucleic Acid Extraction

Synechocystis genomic DNA was isolated for PCR analysis using Qiagen's Plant DNA purification kit (Qiagen, USA) according to the manufacture's protocol.

Generation of ΔCPC-Transformant of Synechocystis sp. PCC6803

A 1,928 bp DNA construct was synthesized (DNA2.0, USA) containing 550 bp of homologous DNA regions upstream and downstream of the CPC-operon, designed to replace the coding region of the CPC-operon with a codon-optimized NPTI gene conferring kanamycin resistance to transformants.

Transformations of Synechocystis were carried out according to procedures established in this lab (Lindberg et al. 2010; Bentley and Melis 2012). Successful replacement of the CPC-operon with the NPTI construct and complete cyanobacterial DNA copy segregation was verified by genomic DNA PCR analysis, using primers further upstream and downstream of the regions of the CPC-operon that were used for homologous recombination, and also by using primers within the CPC-operon (primer sequences are reported in Table 1).

### Pigment Analysis and Biomass Quantification

Chlorophyll a and carotenoid concentrations in cultures were determined spectrophotometrically in 100% methanol extracts of the cells according to Lichtenthaler (1987). Culture biomass accumulation was measured gravimetrically as dry cell weight (dcw), whereby 5 or 10 mL aliquots of a culture were filtered through 0.22 μm Millipore filters and the immobilized cells dried at 100° C. for 12 h prior to weighing the dry cell weight.

### Measurement of Photosynthetic Activity

The oxygen evolution activity of the cultures was measured at 25° C. with a Clark-type oxygen electrode (Rank Brothers, Cambridge, England) illuminated with actinic light from a quartz halogen lamp projector. A Corning 3-69 filter (510 nm cut-off filter, Corning, N.Y.) defined the yellow actinic excitation via which photosynthesis measurements were made. Samples of 5 ml cell suspension containing 1.3 μM Ch1 were loaded onto the oxygen electrode chamber. Sodium bicarbonate (100 μl of 0.5 M solution, pH 7.4) was added to the cell suspension prior to the oxygen evolution measurements to ensure that photosynthesis and oxygen evolution would not be limited by the carbon supply available to the cells. After registration of the rate of dark respiration by the cells, samples were illuminated with gradually increasing light intensities. The rate of oxygen exchange (uptake or evolution) under each of these irradiance conditions was recorded continuously for 2-5 min allowing a linear regression of the slope.

### Cell Fractionation

Cells were harvested by centrifugation at 1,000 g for 3 min at 4° C. Samples were resuspended with ice-cold lysis buffer containing 50 mM Tricine (pH 7.8), 10 mM NaCl, 5 mM MgCl₂, 0.2% polyvinylpyrrolidone-40, 0.2% sodium ascorbate, 1 mM aminocaproic acid, 1 mM aminobenzamidine and 100 μM phenylmethylsulfonyl fluoride (PMSF). Cells were lysed in a French pressure cell operated at 4° C., upon passing twice under 20,000 psi pressure with 30 s cooling intervals on ice. Unbroken cells were removed by centrifugation at 3,000 g for 4 min at 4° C. Membranes were collected by centrifugation of the supernatant at 75,000 g for 45 min at 4° C. The thylakoid membrane pellet was resuspended in a buffer containing 50 mM Tricine (pH 7.8), 10 mM NaCl, 5 mM MgCl₂ for spectrophotometric measurements, or 250 mM Tris-HCl (pH 6.8), 20% glycerol, 7% SDS and 2 M urea for protein analysis.

### SDS-PAGE and Western Blot Analysis

SDS-PAGE and Western blot analyses were performed with total protein from cell extracts, resolved in precast SDS-PAGE “Any KDTM” (BIO-RAD, USA). Loading of samples was based on chlorophyll content and resolved proteins were transferred to a polyvinylidene difluoride (PVDF) membrane (Immobilon-FL 0.45 μm, Millipore, USA) by a tank transfer system. Specific polyclonal antibodies were raised against the spinach D1, D1/D2 PSII reaction center proteins (PSII RC), RBCL, and the ATP-β subunit, as well as the orange carotenoid protein (OCP) from Synechocystis. Cross-reactions were visualized by Supersignal West Pico Chemiluminiscent substrate detection system (Thermo Scientific, USA).

### Spectrophotometric and Kinetic Analyses

The concentration of the photosystems in thylakoid membrane preparations was measured spectrophotometrically from the amplitude of the light-minus-dark absorbance difference signal at 700 nm (P700) for PSI, and 320 nm (Q_{A}) for PSII (Melis and Brown 1980; Melis 1989; Smith et al. 1990). The functional absorption cross-section of PSII and PSI in wild type and transformant Synechocystis and in the respective thylakoid membranes was measured upon weak green actinic excitation (about 50 μmol photons m⁻² s⁻¹) of the samples from the kinetics of Q_{A} photoreduction and P700 photooxidation, respectively (Melis 1989).

### REFERENCES CITED BY AUTHOR AND YEAR IN SPECIFICATION

Ajlani G, Vernotte C (1998) Deletion of the PB-loop in the L_{CM} subunit does not affect phycobilisome assembly or energy transfer functions in the cyanobacterium Synechocystis sp. PCC6714. Eur J Biochem 257: 154-159
Arteni A A, Ajlani G, Boekema E J (2009) Structural organisation of phycobilisomes from Synechocystis sp. strain PCC6803 and their interaction with the membrane. Biochim Biophys Acta 1787: 272-279
Ashby M K and Mullineaux C W (1999) The role of ApcD and ApcF in energy transfer from phycobilisomes to PSI and PSII in a cyanobacterium. Photosynth Res 61: 169.
Bailey S, Grossman A (2008). Photoprotection in cyanobacteria: Regulation of light harvesting. Photochem. Photobiol. 84: 1410-1420
Barber J, Morris E P, da Fonseca P C A (2003) Interaction of the allophycocyanin core complex with photosystem II. Photochem Photobiol Sci 2: 536-541
Bentley F K, Melis A (2012) Diffusion-based process for carbon dioxide uptake and isoprene emission in gaseous/aqueous two-phase photobioreactors by photosynthetic microorganisms. Biotechnol Bioeng 109: 100-109
Bernat G, Waschewski N, Rögner M (2009) Towards efficient hydrogen production: the impact of antenna size and external factors on electron transport dynamics in Synechocystis PCC 6803. Photosynth Res 99: 205-216
Dauville'e D, Delhaye S, Gruyer S, Slomianny C, Moretz S E, d'Hulst C, Long C A, Ball S G, Tomavo S (2010) Engineering the chloroplast targeted malarial vaccine antigens in Chlamydomonas starch granules. PLoS ONE 5: e15424
Ducret A, Sidler W, Wehrli E, Frank G, Zuber H (1996) Isolation, characterization and electron microscopy analysis of a hemidiscoidal phycobilisome type from the cyanobacterium Anabaena sp. PCC 7120. Eur J Biochem 236: 1010-1024
Elmorjani K, Thomas J C, Sebban P (1986) Phycobilisomes of wild type and pigment mutants of the cyanobacterium Synechocystis PCC 6803. Arch. Microbiol., 146 (1986), pp. 186-191
Fujita Y, Murakami A and Ohki K (1987) Regulation of photosystem composition in the cyanobacterial photosynthetic system: The regulation occurs in response to the redox state of the electron pool located between the two photosystems. Plant Cell Physiol 28: 283-292
Glazer A N (1989) Light guides. Directional energy transfer in a photosynthetic antenna. J Biol Chem 264: 1-4.
Glazer A N, Hixson C S (1975) Characterization of R-phycocyanin. Chromophore content of R-phycocyanin and C-phycoerythrin. J Biol Chem 250: 5487-95.
Glazer A N, Melis A (1987) Photochemical reaction centers: structure, organization, and function. Annu Rev Plant Physiol 38: 11-45
Glick R E, Melis A (1988) Minimum photosynthetic unit size in system-I and system-II of barley chloroplasts. Biochim Biophys Acta 934: 151-155
Greenwell H C, Laurens L M L, Shields R J, Lovitt R W, Flynn K J (2010) Placing microalgae on the biofuels priority list: a review of the technological challenges. J R Soc Interface 7: 703-726
Grossman A R, Schaefer M R, Chiang G G, Collier J L (1993) The phycobilisome, a light-harvesting complex responsive to environmental conditions. Microbiol Rev 57: 725-749
Hankamer B, Lehr F, Rupprecht J, Mussgnug J H, Posten C, Kruse O (2007) Photosynthetic biomass and H₂ production by green algae: from bioengineering to bioreactor scale-up. Physiol Plant 131: 10-21
Houmard J, Capuano V, Colombano M V Coursin T, Tandeau de Marsac N (1990) Molecular characterization of the terminal energy acceptor of cyanobacterial phycobilisomes. Proc Natl Acad Sci USA 87: 2152-2956
Hu Q, Sommerfeld M, Jarvis E, Ghirardi M, Posewitz M, Seibert M, Darzins A (2008) Microalgal triacylglycerols as feedstocks for biofuel production: perspectives and advances. Plant J 54: 621-639
Jordan P, Fromme P. Witt H T, Klukas O, Saenger W, Krauβ N (2001) Three-dimensional structure of cyanobacterial photosystem I at 2.5 Å resolution. Nature 411: 909-917
Joseph A, Aikawa S, Sasaki K, Matsuda F, Hasunuma T, Kondo A (2014) Increased biomass production and glycogen accumulation in apcE gene deleted Synechocystis sp. PCC 6803 AMB Express 4: 17
Kirilovsky D (2007) Photoprotection in cyanobacteria: the orange carotenoid protein (OCP)-related non-photochemical-quenching mechanism. Photosynth Res 93: 7-16
Kirilovsky D, Kerfeld C A (2012) The orange carotenoid protein in photoprotection of photosystem II in cyanobacteria Biochim. Biophys Acta 1817: 158-16
Kirst H, Garcia-Cerdin J G, Zurbriggen A, Melis A (2012a) Assembly of the light-harvesting chlorophyll antenna in the green alga Chlamydomonas reinhardtii requires expression of the TLA2-CpFTSY gene. Plant Physiol 158: 930-945
Kirst H, Garcia-Cerdin J G, Zurbriggen A, Rühle T, Melis A (2012b) Truncated photosystem chlorophyll antenna size in the green microalga Chlamydomonas reinhardtii upon deletion of the TLA3-CpSRP43 gene. Plant Physiol 160: 2251-2260
Kirst H, Melis A (2014) The chloroplast signal recognition particle (CpSRP) pathway as a tool to minimize chlorophyll antenna size and maximize photosynthetic productivity. Biotechnol Adv 32: 66-72.
Kwon J H, Bernit G, Wagner H, Rögner M, Rexrotha S (2013) Reduced light-harvesting antenna: Consequences on cyanobacterial metabolism and photosynthetic productivity. Algal Research 2: 188-195
Lichtenthaler H K (1987). Chlorophylls and carotenoids, the pigments of photosynthetic biomembranes. In: Methods Enzymol., 148, Eds. R. Douce and L. Packer. Academic Press Inc., New York, 350-382
Liberton M, Collins A M, Page L E, O'Dell W O, O'Neill H, Urban W S, Timlin J A, Pakrasi H B (2013) Probing the consequences of antenna modification in cyanobacteria Photosyn Res 118: 17-24
MacColl R (1998) Cyanobacterial phycobilisomes. J Struct Biol 124:311-334
Manodori A, Alhadeff M, Glazer A N, Melis A (1984) Photochemical apparatus organization in Synechococcus 6301 (Anacystis nidulans): effect of phycobilisome mutation. Arch. Microbiol. 139: 117-123
Mata T M, Martins A A, Caetano N S (2010) Microalgae for biodiesel production and other applications: a review. Renew Sustain Energy Rev 14: 217-232
Mayfield S P, Manuell A L, Chen S, Wu J, Tran M, Siefker D, Muto M, Marin-Navarro J (2007) Chlamydomonas reinhardtii chloroplasts as protein factories. Curr Opin Biotechnol 18: 126-133
Melis A (1989) Spectroscopic methods in photosynthesis: photosystem stoichiometry and chlorophyll antenna size. Philos Trans R Soc Lond, B 323: 397-409
Melis A (1991) Dynamics of photosynthetic membrane composition and function. Biochim Biophys Acta 1058: 87-106
Melis A (1999) Photosystem-II damage and repair cycle in chloroplasts: what modulates the rate of photodamage in vivo? Trends in Plant Science 4: 130-135
Melis A (2009) Solar energy conversion efficiencies in photosynthesis: minimizing the chlorophyll antennae to maximize efficiency. Plant Science 177: 272-280
Melis A (2007) Photosynthetic H2 metabolism in Chlamydomonas reinhardtii (unicellular green algae). Planta 226: 1075-1086
Melis A (2012) Photosynthesis-to-fuels: from sunlight to hydrogen, isoprene, and botryococcene production. Energy Environ Sci 5:5531-5539
Melis A, Brown J S (1980) Stoichiometry of system I and system II in different photosynthetic membranes. Proc Natl Acad Sci USA 77: 4712-4716Michelet L, Lefebvre-Legendre L, Burr S E, Rochaix J D, Goldschmidt-Clermont M (2011) Enhanced chloroplast transgene expression in a nuclear mutant of Chlamydomonas. Plant Biotechnol J 9: 565-574
Mullineaux C W (2008) Phycobilisome-reaction centre interaction in cyanobacteria. Photosynth Res 95: 175-182
Müller P, Li X P, Niyogi K K (2001) Non-photochemical quenching: a response to excess light energy. Plant Physiol 125: 1558-1566
Myers J. Graham J R, Wang R T K (1980) Light harvesting in Anacystis nidulans studied in pigment mutants. Plant Physiol 66: 1144-1149
Nakajima Y, Ueda R (1997) Improvement of photosynthesis in dense microalgal suspension by reduction of light harvesting pigments. J Appl Phycol 9: 503-510
Nakajima Y, Ueda R (1999) Improvement of microalgal photosynthetic productivity by reducing the content of light harvesting pigments. J Appl Phycol 11: 195-201
Page L E, Liberton M, Pakrasi H (2012) Reduction of photoautotrophic productivity in the cyanobacterium Synechocystis sp. strain PCC 6803 by phycobilisome antenna truncation. Appl Environ Microbiol 78:6349-6351
Sidler W A (1994) Phycobilisome and phycobiliprotein structures. In: Bryant D A (ed) The molecular biology of cyanobacteria. Kluwer Academic, Dordrecht, pp 139-216
Smith B M, Morrissey P J, Guenther J E, Nemson J A, Harrison M A, Allen J F, Melis A (1990) Response of the photosynthetic apparatus in Dunaliella salina (green algae) to irradiance stress. Plant Physiol 93: 1433-1440
Stephens E, Ross I., King Z, Mussgnug J H, Kruse O, Posten C, Borowitzka M A, Hankamer B (2010) An economic and technical evaluation of microalgal biofuels. Nat Biotechnol 28: 126-128
Ughy B, Ajlani G (2004) Phycobilisome rod mutants in Synechocystis sp. strain PCC6803 Microbiology 150: 4147-4156
Umena Y, Kawakami K, Shen J R, Kamiya N (2011) Crystal structure of oxygen-evolving photosystem II at a resolution of 1.9 Å. Nature 473: 55-60

### Illustrative Sequences

| CPC-operon Synechocystis sp. PCC 6803 | |
|---|---|
| SEQ ID NO: 1 | |
| ATGTTCGACGTATTCACTCGGGTTGTTTCCCAAGCTGATGCTCGCGGCGAGTACCTCTCTGG | |
| TTCTCAGTTAGATGCTTTGAGCGCTACCGTTGCTGAAGGCAACAAACGGATTGATTCTGTTA | |
| ACCGCATCACCGGTAATGCTTCCGCTATCGTTTCCAACGCTGCTCGTGCTTTGTTCGCCGAA | |
| CAGCCCCAATTAATCCAACCCGGTGGAAACGCCTACACCAGCCGTCGTATGGCTGCTTGTTT | |
| GCGTGACATGGATATCATCCTCCGCTATGTTACCTACGCAACCTTCACCGGCGACGCTTCCG | |
| TTCTAGAAGATCGTTGCTTGAACGGTCTCCGTGAAACCTACGTTGCCCTGGGTGTTCCCGGT | |
| GCTTCCGTAGCTGCTGGCGTTCAAAAAATGAAAGAAGCTGCCCTGGACATCGTTAACGATCC | |
| CAATGGCATCACCCGTGGTGATTGCAGTGCTATCGTTGCTGAAATCGCTGGTTACTTCGACC | |
| GCGCCGCTGCTGCCGTAGCCTAGTCTGGTTATTTTAAAAACCAACTTTACTCAGGTTCCATA | |
| CCCGAGAAAATCCAGCTTAAAGCTGACATATCTAGGAAAATTTTCACATTCTAACGGGAGAT | |
| ACCAGAACAATGAAAACCCCTTTAACTGAAGCCGTTTCCACCGCTGACTCTCAAGGTCGCTT | |
| TCTGAGCAGCACCGAATTGCAAATTGCTTTCGGTCGTCTACGTCAAGCTAATGCTGGTTTGC | |
| AAGCCGCTAAAGCTCTGACCGACAATGCCCAGAGCTTGGTAAATGGTGCTGCCCAAGCCGTT | |
| TATAACAAATTCCCCTACACCACCCAAACCCAAGGCAACAACTTTGCTGCGGATCAACGGGG | |
| TAAAGACAAGTGTGCCCGGGACATCGGCTACTACCTCCGCATCGTTACCTACTGCTTAGTTG | |
| CTGGTGGTACCGGTCCTTTGGATGAGTACTTGATCGCCGGTATTGATGAAATCAACCGCACC | |
| TTTGACCTCTCCCCCAGCTGGTATGTTGAAGCTCTGAAATACATGAAAGCTAACCACGGCTT | |
| GAGTGGCGATGCCCGTGACGAAGCTAATTCCTACCTCGATTACGCCATCAATGCTCTGAGCT | |
| AGTCAGTTTTTAATTCTAGCTGGCCTGGAAGGGTGGGAAAGTGTTAACAACTGGTTGACAAT | |
| ATTCCGCTTTTCCAGGTCTTGTCGTATTTATTGACACTTATCTAGGAGAACAAAATGACTAG | |
| TTTAGTTTCGGCCCAGCGTCTGGGCATTGTGGCCGTGGATGAGGCTATTCCCCTCGAGCTTC | |
| GTTCCCGGAGTACAGAGGAAGAGGTGGATGCCGTTATCCTGGCGGTTTACCGTCAAGTTTTG | |
| GGCAACGATCATCTCATGTCCCAGGAACGACTGACCAGTGCAGAATCTTTGCTCCGGGGCAG | |
| GGAGATTTCGGTAAGGGATTTTGTTCGGGCTGTGGCTCTGTCGGAAGTGTACCGGCAGAAGT | |
| TTTTCCATTCCAACCCACAAAATCGTTTTATCGAGCTTAATTATAAGCATTTACTGGGACGG | |
| GCTCCCTACGATCAGTCGGAAATTGCTTTCCACACCGATCTTTATCACCAGGGGGGCTATGA | |
| AGCGGAGATCAATTCCTATATTGACTCCGTAGAATACACGGAAAACTTTGGAGATTGGGTTG | |
| TACCTTATTTCCGCGGCTTTGCTACCCAGCGGAATCAAAAAACCGTTGGTTTCAGCCGTTCT | |
| TTCCAGGTGTATCGGGGCTACGCTACCAGTGATCGTTCCCAGGGAAATGGTTCCCGTTCTCG | |
| ATTGACGCGGGAGTTGGCTCGCAATACAGCTTCTCCGGTGTATGCCGGTTCTACGGCCGAGA | |
| GTTTGCGGGGAACTTCGGCGGGTAGTCGTAACCAAATGTATCGTTTGCAAGTGATTCAAGGG | |
| GCGGCCCCCGGTCGTGGCACCCGAGTGCGCCGTGGTAAAGCGGAGTATCTAGTTTCCTACGA | |
| TAATTTGTCTGCCAAGTTGCAACAAATCAATCGCCAAGGAGATACGGTCACAATGATTTCCC | |
| TTGCCTAGAATCAGGATTTTTTGTCAACCTAGTTAGTAAAAGGAGATTATTTAGTGGCAATT | |
| ACAACCGCAGCCTCCAGATTGGGGGTGGCTCCCTATAATGAGTCCCGTCCTGTGGAGTTACG | |
| CCCTGACTTTAGCCTTGATGATGCCAAAATGGTGATTCGGGCTGTATATCGTCAGGTTTTAG | |
| GCAACGATTACATTATGGATTCTGAGCGCCTCAAGGGTGCCGAGTCTCTTTTGACCTATGGT | |
| TCTATTAGTGTGCGGGAATTTGTCCGTACCGTGGCCAAGTCGGAACTTTATAAGAAGAAATT | |
| CCTTTATAACAACTTCCAAACCCGGGTCATTGAACTCAACTACAAACATCTGTTGGGTCGTG | |
| CTCCTTTCAGTGAAGATGAGGTGATTTTCCATCTCGATCTCTATGAAAATCAGGGTTTTGAT | |
| GCGGATATCGATTCTTACATTGATTCGGTCGAATACCAAGAGAACTTTGGGGAAAATATTGT | |
| TCCCTATTATCGTTTTAATAATCAGGTAGGCGATCGCACTGTGGGATTTACCCGCATGTTCC | |
| GTCTTTACCGTGGTTATGCTAACAGTGACCGTTCCCAGTTAGAGCGCAGTTCTAGTCGTCTG | |
| GCAACGGAGTTGGGACAAAATACTGTTTCGGCGATCGTGGGCCCCAGTGGCAGTAATGCTGG | |
| TTGGGCCTATCGTCCTTCTCGGGCCGGCAATACCCCAGCAAAAGCGTTGGGTGGTACGGTTC | |
| CCTTTGGTCAAGCAAGTAAGCTGTTCCGGGTGGAAATTACAGCCATTTCTGCGCCCGGTTAT | |
| CCCAAAGTGCGTCGGAGCAATAAGGCGGTAATTGTTCCCTTTGAACAACTCAACCAGACCTT | |
| ACAACAAATCAATCGTTTGGGGGGTAAGGTTGCTAGTATTACCCCGGCTAGCTTGAGCTAGA | |
| TTTCTGTTTAGTTCAGGAGGAGAAGTGGCTTGACTTTATTTTCCCTTTGGGGAATAACTTGG | |
| GGTCGCTTCTCTTTTTGTCTTTGTAGATGATTTTGCTAATTAGCAAATTAAAACTTTAGTGT | |
| TCAGGAGTTTTTAGAATCGCCATGTTAGGTCAATCTTCTTTGGTGGGCTATTCCAATACCCA | |
| GGCGGCTAACCGTGTCTTTGTCTATGAAGTCAGTGGACTCCGCCAGACCGATGCCAATGAAA | |
| ACAGTGCCCACGATATTCGTCGCAGCGGTAGTGTGTTTATCAAAGTTCCCTATGCCCGTATG | |
| AATGATGAGATGCGCCGCATTTCCCGTCTGGGGGGAACTATTGTTAACATTCGTCCCTATCA | |
| AGCTGACAGCAACGAACAAAACTAG | |
| Synechocystis sp. PCC 6803 CPCA, accession number NP440551.1 | |
| SEQ ID NO: 2 | |
| 1 | mktplteavs tadsqgrfls stelqiafgr lrqanagiqa akaltdnaqs lvngaaqavy |
| 61 | nkfpyttqtq gnnfaadqrg kdkcardigy ylrtvtyclv aggtgpldey liagideinr |
| 121 | tfdlspswyv ealkyikanh glsgdardea nsyidyaina ls |
| Synechocystis sp. PCC 6803 CPCB, accession number NP440552.1 | |
| SEQ ID NO: 3 | |
| 1 | mfdvftrvvs qadargeyls gsqldalsat vaegnkrids vnritgnasa lvsnaaralf |
| 61 | aeqpqliqpg gnaytsrrma aclrdmeiil ryvtyatftg dasvledrcl nglretyval |
| 121 | gvpgasvaag vqkmkeaald ivndpngitr gdcsaivaei agyfdraaaa va |
| Synechocystis sp. PCC 6803 CPCC1, accession number NP440549.1 | |
| SEQ ID NO: 4 | |
| 1 | maittaasrl gvapynesrp velrpdfsld dakmviravy rqvlgndyim dserlkgaes |
| 61 | lltngsisvr efvrtvakse lykkkflynn fqtrvielny khilgrapfs edevifhidl |
| 121 | yenqgfdadi dsyidsveyq enfgenivpy yrfnnqvgdr tvgftrmfrl yrgyansdrs |
| 181 | qlersssrla telgqntvsa ivgpsgsnag wayrpsragn tpakalggtv pfgqasklfr |
| 241 | veitaisapg ypkvrrsnka vivpfeqlnq tlqqinrlgg kvasitpasl s |
| Syncchocystis sp. PCC 6803 CPCC2, accession number NP440550.1 | |
| SEQ ID NO: 5 | |
| 1 | mtelvsaqrl givavdeaip lelrsrstee evdavilavy rgvlgndhlm sqerltsaes |
| 61 | llrgreisvr dfvravalse vyrqkffhsn pqnrfielny khllgrapyd qseiafhtdl |
| 121 | yhqggyeaei nsyidsveyt enfgdwvvpy frgfatqrnq ktvgfarsfq vyrgyatsdr |
| 181 | sqgngsrsrl trelarntas pvyagstaes lrgtsagsrn qmyrlqviqg aapgrgtrvr |
| 241 | rgkaeylvsy dnlsakiqqi nrqgdtvtmi sla |
| Synechocystis sp. PCC 6803 CPCD, accession number NP440548.1 | |
| SEQ ID NO: 6 | |
| 1 | mlgqsslvgy sntqaanrvf vyevsglrqt daneneahdi rrsgsvfikv pyarmndemr |
| 61 | risrlggtiv nirpyqadsn eqn |

1. A method of enhancing photosynthetic productivity from a cyanobacteria, the method comprising:
providing a cyanobacterial cell population in which the cyanobacteria are genetically modified to have a disruption in a CPC-operon;
growing the cyanobacterial cell population in a reactor to obtain a cyanobacterial culture;
maintaining the culture under conditions in which the light intensity is at least 500 micromol photons per square meter per second; and the culture absorbs at least 70% of the incoming light.

2. The method of claim 1, wherein the light intensity is at least 800 micromol photons per square meter per second.

3. The method of claim 1, wherein the culture absorbs at least 80%, or at least 90% of the incoming light.

4. The method of claim 1, wherein the disruption in the CPC-operon is inhibition of a CPCA and/or CPCB gene.

5. The method of claim 1, wherein the disruption in the CPC-operon is inhibition of at least one CPCC1, CPCC2, and CPCD.

6. The method of claim 1, wherein the disruption in the CPC-operon is a deletion of a CPCA, CPCB, CPCC1, CPCC2, and CPCD genes.

7. The method of claim 1, wherein the cyanobacteria are of a species a genus selected from the group consisting of Synechocystis, Synechococcus, Cyanothece, and Thermosynechococcus.

8. The method of claim 1, wherein the cyanobacteria are of a species of a genus of filamentous cyanobacteria.

9. The method of claim 8, wherein the genus is selected from the group consisting of Arthrospira, Nostoc, and Anabaena.


SEQUENCE LISTING (14 sequences)

SEQ ID NO: 1 - DNA, 3373 bp, Synechocystis sp. PCC6803
atgttcgacgtattcactcgggttgtttcccaagctgatgctcgcggcgagtacctctctggttctcagttagatgctttgagcgctaccgttgctgaaggcaacaaacggattgattctgttaaccgcatcaccggtaatgcttccgctatcgtttccaacgctgctcgtgctttgttcgccgaacagccccaattaatccaacccggtggaaacgcctacaccagccgtcgtatggctgcttgtttgcgtgacatggaaatcatcctccgctatgttacctacgcaaccttcaccggcgacgcttccgttctagaagatcgttgcttgaacggtctccgtgaaacctacgttgccctgggtgttcccggtgcttccgtagctgctggcgttcaaaaaatgaaagaagctgccctggacatcgttaacgatcccaatggcatcacccgtggtgattgcagtgctatcgttgctgaaatcgctggttacttcgaccgcgccgctgctgccgtagcctagtctggttattttaaaaaccaactttactcaggttccatacccgagaaaatccagcttaaagctgacatatctaggaaaattttcacattctaacgggagataccagaacaatgaaaacccctttaactgaagccgtttccaccgctgactctcaaggtcgctttctgagcagcaccgaattgcaaattgctttcggtcgtctacgtcaagctaatgctggtttgcaagccgctaaagctctgaccgacaatgcccagagcttggtaaatggtgctgcccaagccgtttataacaaattcccctacaccacccaaacccaaggcaacaactttgctgcggatcaacggggtaaagacaagtgtgcccgggacatcggctactacctccgcatcgttacctactgcttagttgctggtggtaccggtcctttggatgagtacttgatcgccggtattgatgaaatcaaccgcacctttgacctctcccccagctggtatgttgaagctctgaaatacatcaaagctaaccacggcttgagtggcgatgcccgtgacgaagctaattcctacctcgattacgccatcaatgctctgagctagtcagtttttaattctagctggcctggaagggtgggaaagtgttaacaactggttgacaatattccgcttttccaggtcttgtcgtatttattgacacttatctaggagaacaaaatgactagtttagtttcggcccagcgtctgggcattgtggccgtggatgaggctattcccctcgagcttcgttcccggagtacagaggaagaggtggatgccgttatcctggcggtttaccgtcaagttttgggcaacgatcatctcatgtcccaggaacgactgaccagtgcagaatctttgctccggggcagggagatttcggtaagggattttgttcgggctgtggctctgtcggaagtgtaccggcagaagtttttccattccaacccacaaaatcgttttatcgagcttaattataagcatttactgggacgggctccctacgatcagtcggaaattgctttccacaccgatctttatcaccaggggggctatgaagcggagatcaattcctatattgactccgtagaatacacggaaaactttggagattgggttgtaccttatttccgcggctttgctacccagcggaatcaaaaaaccgttggtttcagccgttctttccaggtgtatcggggctacgctaccagtgatcgttcccagggaaatggttcccgttctcgattgacgcgggagttggctcgcaatacagcttctccggtgtatgccggttctacggccgagagtttgcggggaacttcggcgggtagtcgtaaccaaatgtatcgtttgcaagtgattcaaggggcggcccccggtcgtggcacccgagtgcgccgtggtaaagcggagtatctagtttcctacgataatttgtctgccaagttgcaacaaatcaatcgccaaggagatacggtcacaatgatttcccttgcctagaatcaggattttttgtcaacctagttagtaaaaggagattatttagtggcaattacaaccgcagcctccagattgggggtggctccctataatgagtcccgtcctgtggagttacgccctgactttagccttgatgatgccaaaatggtgattcgggctgtatatcgtcaggttttaggcaacgattacattatggattctgagcgcctcaagggtgccgagtctcttttgaccaatggttctattagtgtgcgggaatttgtccgtaccgtggccaagtcggaactttataagaagaaattcctttataacaacttccaaacccgggtcattgaactcaactacaaacatctgttgggtcgtgctcctttcagtgaagatgaggtgattttccatctcgatctctatgaaaatcagggttttgatgcggatatcgattcttacattgattcggtcgaataccaagagaactttggggaaaatattgttccctattatcgttttaataatcaggtaggcgatcgcactgtgggatttacccgcatgttccgtctttaccgtggttatgctaacagtgaccgttcccagttagagcgcagttctagtcgtctggcaacggagttgggacaaaatactgtttcggcgatcgtgggccccagtggcagtaatgctggttgggcctatcgtccttctcgggccggcaataccccagcaaaagcgttgggtggtacggttccctttggtcaagcaagtaagctgttccgggtggaaattacagccatttctgcgcccggttatcccaaagtgcgtcggagcaataaggcggtaattgttccctttgaacaactcaaccagaccttacaacaaatcaatcgtttggggggtaaggttgctagtattaccccggctagcttgagctagatttctgtttagttcaggaggagaagtggcttgactttattttccctttggggaataacttggggtcgcttctctttttgtctttgtagatgattttgctaattagcaaattaaaactttagtgttcaggagtttttagaatcgccatgttaggtcaatcttctttggtgggctattccaatacccaggcggctaaccgtgtctttgtctatgaagtcagtggactccgccagaccgatgccaatgaaaacagtgcccacgatattcgtcgcagcggtagtgtgtttatcaaagttccctatgcccgtatgaatgatgagatgcgccgcatttcccgtctggggggaactattgttaacattcgtccctatcaagctgacagcaacgaacaaaactag

SEQ ID NO: 2 - protein, 162 aa, Synechocystis sp. PCC 6803 CPCA
MKTPLTEAVSTADSQGRFLSSTELQIAFGRLRQANAGLQAAKALTDNAQSLVNGAAQAVYNKFPYTTQTQGNNFAADQRGKDKCARDIGYYLRIVTYCLVAGGTGPLDEYLIAGIDEINRTFDLSPSWYVEALKYIKANHGLSGDARDEANSYLDYAINALS

SEQ ID NO: 3 - protein, 172 aa, Synechocystis sp. PCC 6803 CPCB
MFDVFTRVVSQADARGEYLSGSQLDALSATVAEGNKRIDSVNRITGNASAIVSNAARALFAEQPQLIQPGGNAYTSRRMAACLRDMEIILRYVTYATFTGDASVLEDRCLNGLRETYVALGVPGASVAAGVQKMKEAALDIVNDPNGITRGDCSAIVAEIAGYFDRAAAAVA

SEQ ID NO: 4 - protein, 291 aa, Synechocystis sp. PCC 6803 CPCC1
MAITTAASRLGVAPYNESRPVELRPDFSLDDAKMVIRAVYRQVLGNDYIMDSERLKGAESLLTNGSISVREFVRTVAKSELYKKKFLYNNFQTRVIELNYKHLLGRAPFSEDEVIFHLDLYENQGFDADIDSYIDSVEYQENFGENIVPYYRFNNQVGDRTVGFTRMFRLYRGYANSDRSQLERSSSRLATELGQNTVSAIVGPSGSNAGWAYRPSRAGNTPAKALGGTVPFGQASKLFRVEITAISAPGYPKVRRSNKAVIVPFEQLNQTLQQINRLGGKVASITPASLS

SEQ ID NO: 5 - protein, 273 aa, Synechocystis sp. PCC 6803 CPCC2
MTSLVSAQRLGIVAVDEAIPLELRSRSTEEEVDAVILAVYRQVLGNDHLMSQERLTSAESLLRGREISVRDFVRAVALSEVYRQKFFHSNPQNRFIELNYKHLLGRAPYDQSEIAFHTDLYHQGGYEAEINSYIDSVEYTENFGDWVVPYFRGFATQRNQKTVGFSRSFQVYRGYATSDRSQGNGSRSRLTRELARNTASPVYAGSTAESLRGTSAGSRNQMYRLQVIQGAAPGRGTRVRRGKAEYLVSYDNLSAKLQQINRQGDTVTMISLA

SEQ ID NO: 6 - protein, 83 aa, Synechocystis sp. PCC 6803 CPCD
MLGQSSLVGYSNTQAANRVFVYEVSGLRQTDANENSAHDIRRSGSVFIKVPYARMNDEMRRISRLGGTIVNIRPYQADSNEQN

SEQ ID NO: 7 - DNA, 34 bp, Artificial Sequence
  synthetic primer
gacttgaatgtcactaactacatccagtctttgc

SEQ ID NO: 8 - DNA, 28 bp, Artificial Sequence
  synthetic primer
gctaaatcccatgaagagaaggtttatg

SEQ ID NO: 9 - DNA, 24 bp, Artificial Sequence
  synthetic primer
ccattagcaaggcaaatcaaagac

SEQ ID NO: 10 - DNA, 23 bp, Artificial Sequence
  synthetic primer
ggtggaaacggcttcagttaaag

SEQ ID NO: 11 - DNA, 24 bp, Artificial Sequence
  synthetic primer
gttccctttggtcaagcaagtaag

SEQ ID NO: 12 - DNA, 30 bp, Artificial Sequence
  synthetic primer
ggttgattcgtttacatcagttcaataaag

SEQ ID NO: 13 - DNA, 25 bp, Artificial Sequence
  synthetic primer
ccattaaacattgtgcttacactcc

SEQ ID NO: 14 - DNA, 28 bp, Artificial Sequence
  synthetic primer
gaagtgccagtgactaacctttatcgag